(19) United States
(12) Patent Application Publication
(10) Pub. No.: US 2017/0067110 A1
(43) Pub. Date: Mar. 9, 2017

(54) DETECTION METHOD FOR GENETIC DISEASE

(71) Applicant: KAGOSHIMA UNIVERSITY, Kagoshima-shi, Kagoshima (JP)

(72) Inventors: Hiroshi TAKASHIMA, Kagoshima (JP); Yujiro HIGUCHI, Kagoshima (JP); Akihiro HASHIGUCHI, Kagoshima (JP); Akiko YOSHIMURA, Kagoshima (JP)

(73) Assignee: KAGOSHIMA UNIVERSITY, Kagoshima-shi, Kagoshima (JP)

(21) Appl. No.: 15/307,061

(22) PCT Filed: Apr. 27, 2015
(86) PCT No.: PCT/JP2015/062701

§ 371 Date: Oct. 27, 2016

(30) Foreign Application Priority Data
Apr. 28, 2014 (JP) 2014-093044

(51) Int. Cl. C12Q 1/68

(52) U.S. Cl. CPC C12Q 1/6883; C12Q 2600/156

(57) ABSTRACT

The present invention relates to a detection method for a genetic disease, and specifically relates to detection of disease-causing genes for autosomal recessive inherited Charcot-Marie-Tooth disease (CMT). In the method according to the present invention, a mutation(s) in MME (membrane metallo-endopeptidase) gene, FAT3 (FAT tumor suppressor homolog 3) gene, and/or SELRC1 (Sell repeat containing 1) gene in a biological sample are/is detected.

### TECHNICAL FIELD

The present invention relates to a detection method for a genetic disease, and specifically relates to detection of disease-causing genes for autosomal recessive inherited Charcot-Marie-Tooth disease (CMT).

Research relating to the present invention was conducted under approval of Ethics Committee of Kagoshima University Faculty of Medicine, and informed consent had been obtained from all patients who were subjected to a genetic test and their family members and they had expressed written consent to participate in the research.

### BACKGROUND ART

Charcot-Marie-Tooth disease (CMT), which is also referred to as Hereditary Motor Sensory Neuropathy (HMSN), is the most representative genetic disease among hereditary neuropathies. Its clinical characteristics include progressive muscle weakness that begins in the distal limb muscle, foot deformity, sensory disturbance, and decreased or absent deep tendon reflexes.

In clinical genetics, demyelinating CMT caused by disorders of myelin and axonal CMT caused by disorders of axons among autosomal dominant inherited CMTs are classified as CMT1 and CMT2, respectively, and demyelinating CMT caused by disorders of myelin and axonal CMT caused by disorders of axons among autosomal recessive inherited CMTs are classified as CMT4 (AR-CMT1) and AR-CMT2, respectively, and X-linked CMT is classified as CMTX. Demyelinating CMT and axonal CMT can be discriminated by using a threshold of 38 m/sec of motor nerve conduction velocity (MCV) in the median nerve.

In addition, demyelinating CMT is clinically classified on the basis of age of onset and severity. The severest CMT which causes congenital floppy infant is classified as congenital hypomyelinating neuropathy (CHN), and CMT which develops at a stage between birth to the infant stage (typically, two years old or younger) is classified as Dejerine-Sottas syndrome (DSS). Further, CMT which produces an MCV between demyelinating CMT and axonal CMT is called as intermediate CMT. Thus, CMT is a diverse disease genetically and also clinically.

Since Lupski et al. reported duplication of PMP22 gene as a cause for CMT1A in 1991, at least 40 CMT-causing genes have been reported, and for example, FIG. 4 gene has been reported as one of CMT4-causing genes (Patent Literature 1). In combination with CMT-related diseases including hereditary motor neuropathy (HMN), hereditary sensory neuropathy (HSN), and hereditary sensory and autonomic neuropathy (HSAN), the number of CMT-causing genes has reached 65 or more. Proteins encoded by currently reported CMT-causing genes are known to have various functions, such as (i) a myelin-component protein, (ii) a transcription factor for a myelin-related protein, (iii) transportation, metabolism, and processing of a myelin-related protein, (iv) cell differentiation and cell maintenance, (v) a function relating to transportation of neurofilaments and proteins, (vi) a function relating to mitochondria, (vii) repair and transcription of DNA and synthesis of a nucleic acid, (viii) an ion channel, and (iv) aminoacyl-tRNA synthetase.

Currently, duplication tests for PMP22 gene which provide an indicator of CMT1A are conducted by a commercial laboratory with the support of the National Health Insurance in Japan. For a part of other known causal genes, Athena diagnostics, U.S.A., commercially provides genetic test, and Kagoshima University conducts genetic test in Japan.

On the other hand, progress in the genomic analysis technique through Next-Generation Sequencing (NGS) since 2005 has enabled faster gene analysis at a lower cost. Since a causal gene for Mendelian genetic diseases was identified by using exome analysis for the first time in 2010, a large number of pathological mutations relating to human genetic diseases have been identified, and new causal genes for CMT have been also discovered (Non Patent Literatures 1 to 4).

### CITATION LIST

### Patent Literature

Patent Literature 1: JP Patent Publication (Kohyo) No. 2010-525819A

### Non Patent Literature

Non Patent Literature 1: Montenegro, G. et al., Ann. Neurol. 69, 464-70 (2011)
Non Patent Literature 2: Soong, B. W. et al., Am. J. Hum. Genet. 92, 422-30 (2013)
Non Patent Literature 3: Ylikallio, E. et al., Hum. Mol. Genet., 22, 2975-83 (2013)
Non Patent Literature 4: Kennerson, M. L. et al., Hum. Mol. Genet. 22, 1404-16 (2013)

### SUMMARY OF INVENTION

### Technical Problem

The CMT incidence is considered to be approximately 1/2,500, and incidences for different types of CMT have been reported in part. However, not all of the causal genes have been identified, and thus the percentage of cases the cause of which can be identified by using current genetic diagnosis is 70% or less, except the most frequent CMT1A cases (e.g., PMP22 duplication). Further, demyelinating CMT1A incidence is low in Japan, in contrast to the Western countries, and the positive ratio in the test is significantly low for axonal and intermediate CMTs.

In addition, the reported causal genes have various functions, and the mechanism of the diseases has not been revealed and proper treatment method therefor has not been found.

Clinical diagnosis for CMT is comprehensively conducted by neurologists or pediatricians via medical examinations and tests on the basis of clinical course, neurological findings, findings in tests including a blood test, a nerve conduction test, and a neuroimaging test, family history, etc. However, definite diagnosis is difficult because, for example, symptoms presented differ from patient to patient.

There are many diseases which cause symptoms and course similar to those for CMT, and cases of slowly-progressive peripheral polyneuropathy are all regarded as a subject for differential diagnosis. Examples thereof include chronic inflammatory demyelinating polyneuropathy (CIDP), multifocal motor neuropathy (MMN), diabetic polyneuropathy, and alcoholic neuropathy. CIDP and MMN are each a disease treatable with immunotherapy. In the case of diabetic polyneuropathy and alcoholic neuropathy, proper treatment of underlying diseases and guidance of lifestyle are expected to achieve amelioration of the symptoms and prevention of the progression. CMT patients are often misdiagnosed as such diseases and under a risk of selecting improper treatment. Contemplated is a case that medication, etc., based on incorrect diagnosis worsens the symptoms. It is thus desirable for CMT patients to be provided with definite diagnosis through genetic diagnosis.

Accordingly, it is desired to discover a new causal gene for CMT cases with unknown cause, in particular for axonal and intermediate CMTs, in order to diagnose, prevent, and treat such diseases. Further identification of causal genes enables reduction of anxiety of patients and their family members caused by unknown cause for the diseases and prediction of prognosis, and as a result can lead to selection of a proper medical care and treatment. In addition, it can lead to pathological understanding of the diseases, drug discovery research, and development of a new treatment method including gene therapy.

Purposes of conducting a genetic test for a patient clinically diagnosed as CMT include the following.

1. Definite diagnosis can be provided (reduction of misdiagnosis).
2. The prognosis and complications of the disease can be predicted. For example, cases of CMTX caused by GJB1 mutation associated with hearing loss and central nervous system symptoms are reported (Takashima H, et al.: Acta Neurol Scand 2003) and there is a report that climbing (high-altitude environment) and infections are each an inducing factor for the onset of central nervous system symptoms (Henry L, et al.: Annals of Neurology 2002). Thus, patients can be provided with specific guidance of daily life.
3. Invasive tests such as nerve biopsy can be avoided.
4. Selection of a proper treatment method and an opportunity to participate in a clinical trial can be provided.
For CMT1A patients due to PMP22 duplication, clinical trials with ascorbic acid have been already conducted.

### Solution to Problem

The present inventors extracted cases with unidentified cause from numerous CMT cases, and tried searching a novel causal gene for autosomal recessive inherited CMT from mutation data in exome analysis for an unprecedented number of cases using a uniquely developed “disease candidate gene narrowing-down system”.

The number of mutations detected per case in exome analysis reaches 10,000 to 12,000 even for non-synonymous mutation only, and thus it is really hard to identify a disease gene among them. In such a circumstance, filtering of mutations is the most frequently used method to identify a causal gene. To exclude pseudo-positive mutation calls, mutations are filtered on the basis of quality, minimal read depth, etc., and from the filtered mutations, mutations including common SNPs which are frequently present, rare SNPs registered in databases, and synonymous mutations which cause no amino acid substitution, are excluded to extract only non-synonymous mutations, splicing site mutations, and insertion/deletion mutations. These operations are considered to be capable of narrowing down the number of mutations to approximately 500 to 700. This is merely insufficient, however, and a further strategy is required to find out a disease-related mutation among them.

If a mutation which is not found in healthy individuals and is shared only by patients suffering from an identical disease are present in the residual mutations after filtering, it is expected that the mutation is highly likely to be a pathological mutation. On the basis of this theory, the present inventors used an “overlap strategy” to identify a causal gene. For identification of a causal gene, analysis was performed with sufficient consideration of the mode of inheritance of the disease.

In the case of autosomal dominant inherited diseases, it is needed to search a heterozygous mutation shared only by affected individuals. In the case of only one family, an identical haplotype block is often shared by affected individuals in the family, and thus narrowing down of pathological mutations has limitations. When a causal gene is identified from a plurality of families, identification of a causal gene often works well by searching a gene having a mutation shared among families, if the disease is a monogenic and uniform phenotype (homogeneous) disease. In the case where a disease is a genetically and clinically nonuniform phenotype (heterogeneous) disease as CMT, however, identification of the causal gene is often difficult. The fact that there exist a large number of race-specific rare SNPs is another factor complicating it. Previously, a causal gene could be found out by narrowing down candidate regions using linkage analysis or the like as long as large families having many affected individuals are targeted, even if the number of the families is small. In recent Japan, identification of a causal gene has become more difficult in the case that a few families are targeted because of problems of resources such as smallness of the scale of each family associated with increase in the number of nuclear families and fewer opportunities to receive cooperation for genes from family members living separately.

In the case of autosomal recessive inherited diseases, on the other hand, the onset occurs due to a homozygous mutation or a compound heterozygous mutation. Thus, focusing only on these mutations enables narrowing down of shared mutations from a relatively small number of candidate mutations. Especially in the case that consanguineous marriage is found in the family, searching with focus only on homozygous mutations is considered to be an extremely efficient strategy.

The present inventors, focusing on autosomal recessive inherited CMT, screened numerous, 304, CMT (or hereditary neuropathy) cases to exclude cases with family history of autosomal dominant inherited CMT, and thus obtained 179 cases with unidentified cause. And the present inventors succeeded in simultaneous identification of at least three novel potential causal genes for CMT from the numerous mutation data in exome analysis using the above-mentioned “disease candidate gene narrowing-down system” which had been uniquely developed on the basis of the overlap strategy.

Specifically, the present invention provides the following.

1. A method for acquiring data for diagnosis of autosomal recessive inherited Charcot-Marie-Tooth disease (CMT), wherein a mutation(s) in MME (membrane metallo-endopeptidase) gene, FAT3 (FAT tumor suppressor homolog 3) gene, and/or SELRC1 (Sell repeat containing 1, alias, COAT: cytochrome c oxidase assembly factor 7) gene in a biological sample are/is detected.
2. A method for acquiring data for diagnosis of Charcot-Marie-Tooth disease, wherein a mutation in a DNA in a biological sample is detected, and the mutation is one or more mutations in the nucleotide sequence represented by SEQ ID NO: 1, 3, or 5.
3. The method according to the above 1 or 2, wherein the mutation is any non-synonymous mutation of a missense mutation, a nonsense mutation, and a frameshift mutation.
4. A method for acquiring data for diagnosis of Charcot-Marie-Tooth disease comprising: determining a nucleotide sequence of MME gene in a sample; and comparing the sequence with a nucleotide sequence represented by SEQ ID NO: 1 to determine the presence or absence of a mutation.
5. The method according to the above 4, wherein the mutation is one or more of a mutation on a splice donor site between exons 7 and 8 in MME gene (c.654+1G>A) (a mutation of guanine to adenine at position 37341 of SEQ ID NO: 1), a nonsense mutation on exon 8 in MME gene (c.661C>T, p.Q221X) (a mutation of cytosine to thymine at position 39106 of SEQ ID NO: 1, a mutation of a glutamine residue to a termination codon at position 221 of SEQ ID NO: 2), and a missense mutation on exon 19 in MME gene (c.1861T>C, p.C621R) (a mutation of thymine to cytosine at position 88926 of SEQ ID NO: 1, a mutation of a cysteine residue to an arginine residue at position 621 of SEQ ID NO: 2).
6. A method for acquiring data for diagnosis of Charcot-Marie-Tooth disease comprising:

determining a nucleotide sequence of FAT3 gene in a sample; and

comparing the sequence with a nucleotide sequence represented by SEQ ID NO: 3 to determine the presence or absence of a mutation.

7. The method according to the above 6, wherein the mutation is one or more of a missense mutation on exon 9 in FAT3 gene (c.6122C>A, p.P2041H) (a mutation of cytosine to adenine at position 484856 of SEQ ID NO: 3, a mutation of a proline residue to a histidine residue at position 2041 of SEQ ID NO: 4) and a missense mutation on exon 18 in FAT3 gene (c.11327G>A, p.C3776Y) (a mutation of guanine to adenine at position 530415 of SEQ ID NO: 3, a mutation of a cysteine residue to a tyrosine residue at position 3776 of SEQ ID NO: 4).
8. A method for acquiring data for diagnosis of Charcot-Marie-Tooth disease comprising:

determining a nucleotide sequence of SELRC1 gene in a sample; and

comparing the sequence with a nucleotide sequence represented by SEQ ID NO: 5 to determine the presence or absence of a mutation.

9. The method according to the above 8, wherein the mutation is one or more of a missense mutation on exon 2 in SELRC1 gene (c.115C>T, p.R39W) (a mutation of cytosine to thymine at position 5508 of SEQ ID NO: 5, a mutation of an arginine residue to a tryptophan residue at position 39 of SEQ ID NO: 6) and a missense mutation on exon 1 in SELRC1 gene (c.17A>G, p.D6G) (a mutation of adenine to guanine at position 57 of SEQ ID NO: 5, a mutation of an aspartic acid residue to a glycine residue at position 6 of SEQ ID NO: 6).
10. A primer or probe for detection of autosomal recessive inherited CMT, being a nucleic acid consisting of a nucleotide sequence represented by any of SEQ ID NOs: 1, 3 and 5 or a partial nucleic acid thereof.
11. A DNA chip for detection of autosomal recessive inherited CMT, comprising the probe according to the above 10.
12. A kit for detection of a mutation in MME gene, FAT3 gene, and/or SELRC1 gene in a biological sample to be used in the method according to any one of the above 1 to 9.

The present specification encompasses the contents described in the specification and/or the drawings of JP Patent Application No. 2014-093044, on which the priority of the present application is based.

### Advantageous Effects of Invention

The present invention enables identification of a causal gene for cases which cannot be diagnosed as CMT in conventional genetic diagnosis and prediction of the possibility of future onset for an individual before onset. Further, even in the case of an individual with no possibility of onset due to that the mutation is heterozygous, the present invention enables prediction of the possibility of onset for later generations on the basis of information of having a mutated gene. In addition, it is expected that use of gene therapy with a nonmutated gene prevents the onset and alleviates the symptoms.

Addition of a novel gene as a causal gene enables reduction of anxiety of patients and their family members caused by unknown cause and prediction of the prognosis, and as a result can lead to selection of a proper medical care and treatment, and in addition, can contribute to pathological understanding of the diseases, drug discovery research, and development of a new treatment method including gene therapy.

Although a disease before onset or in the initial stage can be detected, presymptomatic testing should be conducted in accordance with the “GUIDELINES FOR GENETIC TESTING” V.3.A. Presymptomatic Testing, which was established by Genetic-Medicine-Related Societies.

### BRIEF DESCRIPTION OF DRAWINGS

FIG. 1 is a flow chart for screening of cases using a microarray and exome analysis.

FIG. 2 illustrates narrowing down of autosomal recessive inherited mutations using a “disease candidate narrowing-down system”.

FIG. 3 illustrates family histories of five families each having MME gene mutation (patient IDs: 4590, 4229, 4309, 3676, and 4185).

FIG. 4 illustrates a schematic of neprilysin encoded by MME gene and mutation sites.

FIG. 5 illustrates the family history of a patient having an MME gene mutation (patient ID: 3676) and difference in the nucleotide sequence at a splice donor site in MME gene among the family members. A. family history, B. MME gene mutation (c.654+1G>A at splice donor site).

FIG. 6A illustrates the family history of a patient having a FAT3 gene mutation (patient ID: 3743) and difference in the nucleotide sequence of FAT3 gene and the amino acid encoded thereby among the family members. Left: family history, right: FAT3 gene mutation (c.6122C>A, p.P2041H).

FIG. 6B illustrates the family history of a patient having a FAT3 gene mutation (patient ID: 3887) and difference in the nucleotide sequence of FAT3 gene and the amino acid encoded thereby among the family members. Left: family history, right: FAT3 gene mutation (c.11327G>A, p.C3776Y).

FIG. 7A illustrates the family history of a patient having an SELRC1 gene mutation (patient ID: 4040) and difference in the nucleotide sequence of SELRC1 gene and the amino acid encoded thereby among the family members. Left: family history, right: SELRC1 gene mutation (c.17A>G, p.D6G).

FIG. 7B shows interspecies comparison of a partial sequence containing Asp6 in the amino acid sequence encoded by SELRC1 gene.

FIG. 7C shows brain MRI images for a patient having an SELRC1 gene mutation (patient ID: 4040).

FIG. 8A illustrates the family history of a patient having an SELRC1 gene mutation (patient ID: 4348) and difference in the nucleotide sequence of SELRC1 gene and the amino acid encoded thereby among the family members. Left: family history, right: SELRC1 gene mutation (c.115C>T, p. R39W).

FIG. 8B shows interspecies comparison of a partial sequence containing Arg39 in the amino acid sequence encoded by SELRC1 gene.

FIG. 8C shows brain MRI images for a patient having an SELRC1 gene mutation (patient ID: 4348).

### DESCRIPTION OF EMBODIMENTS

### Detection of Mutation in MME Gene, FAT3 Gene, and/or SELRC1 Gene

The present invention is in particular characterized in that a mutation in MME (membrane metallo-endopeptidase) gene, FAT3 (FAT tumor suppressor homolog 3) gene, and/or SELRC1 (Sell repeat containing 1) gene in a biological sample are/is detected.

In the present invention, a “biological sample” refers to a sample derived from a subject, and may be any biological sample containing a DNA, an mRNA, or a protein. It is known in the art that DNA information can be obtained from various minute samples such as tissues, body fluids, and body hairs derived from a subject. Although the sample is not limited, saliva, blood, etc., are suitable in view of a lower burden imposed on a subject in sampling. In particular, the subject is a human, but not limited thereto. Those skilled in the art can easily perform DNA sampling, and for example, can extract a genome DNA from the peripheral blood of a subject by using Gentra Puregene Blood Kit (QIAGEN, Tokyo, Japan).

The present inventors found that a subject having a mutation in any of the above three genes can result in the onset of CMT. The mode of inheritance for all of the three genes is obviously autosomal recessive inheritance, and thus the onset of the symptoms of CMT due to a mutation in the genes occurs if a homozygous mutation or a compound heterozygous mutation is present. The onset occurs because proteins encoded by the genes are not expressed normally or do not function normally, and specific details of mutations found in each patient do not matter. Thus, mutation in the genes to be detected in the present invention is not limited and may be any mutation which does not allow for expression of a functional protein. Examples thereof include non-synonymous mutations such as missense mutations, nonsense mutations, and frameshift mutations, including substitution, deletion, insertion, and duplication of a nucleotide in an exon and mutation at a splicing site; and do not include mutations in an intron and synonymous mutations, which do not involve an amino acid change.

Mutations to be detected in the present invention may be homozygous mutations or compound heterozygous mutations, or heterozygous mutations in which one allele is normal. In a patient after the onset, a homozygous or compound heterozygous mutation is presumably present in the gene. In the case of an individual in whom the onset is expected to occur or a carrier, a mutation may be present in one or both of the alleles. Whether a mutation is homozygous or heterozygous can be determined by using, for example, DNA sequence comparison according to Sanger method.

Mutation in a gene can be detected by analyzing the sequence of a DNA, an mRNA (cDNA), or a protein, as described above.

In one aspect of the present invention, the present invention provides a method for acquiring data for diagnosis of Charcot-Marie-Tooth disease, in which a mutation in a DNA in a biological sample is detected, and the mutation is one or more mutations in the nucleotide sequences represented by SEQ ID NO: 1, 3, and 5.

In the present specification, “acquiring data for diagnosis of Charcot-Marie-Tooth disease” means acquiring data containing a detection result for the presence or absence of a mutation in a gene in a sample, and the data are to serve to assist a physician to diagnose whether the disease is Charcot-Marie-Tooth disease.

### <Mutation in MME Gene>

The MME gene is located in the long arm of human chromosome 3 (3q25.2). Neprilysin (NEP), a protein encoded by the MME gene, is a cell membrane-associated protease which cleaves a peptide linkage of proteins at the amino terminal of a hydrophobic amino acid residue, and also called as Enkephalinase or neutral endopeptidase 24.11 (Turner, A. J., Isaac, R. E. & Coates, D., Bioessays 23, 261-9 (2001)). NEP is expressed in various normal tissues such as kidney, skeletal muscle, central nervous system, peripheral nervous system, skin, etc., and in particular in the central nervous system, NEP is known to be expressed even in pyramidal cells in the cerebral neocortex and the vascular smooth muscle of the cerebral blood vessel. In addition, NEP is a major enzyme to decompose amyloid β peptides (aggregates of abnormal, misfolded proteins), and reduction of NEP activity is known to be associated with the onset of Alzheimer's disease. Thus, NEP is one of enzymes attracting attention as a key molecule in pathological understanding and drug discovery research for Alzheimer's disease.

The nucleotide sequence of the gene encoding the functional MME protein and the amino acid sequence of the MME protein are represented by SEQ ID NOs: 1 and 2, respectively. The positions of exons in the MME gene sequence represented by SEQ ID NO: 1 are listed in the following Table 1.

TABLE 1
Information on exons in MME gene sequence

| Exon | Length (nucleotides) | Position in SEQ ID NO: 1 |
|---|---|---|
| 1*¹ | 110 | 1 to 110 |
| 2*¹ | 170 | 4,512 to 4,681 |
| 3 | 36 | 5,413 to 5,448 |
| 4 | 162 | 35,348 to 35,509 |
| 5 | 81 | 36,833 to 36,913 |
| 6 | 96 | 37,018 to 37,113 |
| 7 | 119 | 37,222 to 37,340 |
| 8 | 66 | 39,100 to 39,165 |
| 9 | 135 | 58,456 to 58,590 |
| 10 | 102 | 60,545 to 60,646 |
| 11 | 137 | 62,345 to 62,481 |
| 12 | 94 | 62,591 to 62,684 |
| 13 | 129 | 63,797 to 63,925 |
| 14 | 99 | 64,713 to 64,811 |
| 15 | 81 | 67,498 to 67,578 |
| 16 | 104 | 68,904 to 69,007 |
| 17 | 59 | 80,744 to 80,802 |
| 18 | 120 | 87,256 to 87,375 |
| 19 | 134 | 88,846 to 88,979 |
| 20 | 66 | 89,086 to 89,151 |
| 21 | 96 | 92,471 to 92,566 |
| 22 | 77 | 92,890 to 92,966 |
| 23*² | 3,349 | 100,714 to 104,062 |

*¹including 5′UTR: 1 to 4,521
*²including 3′UTR: 100,814 to 104,062

If the functional MME protein is not expressed due to mutation in the MME gene, the onset of CMT is expected to occur. Accordingly, one aspect of the present invention is a method for acquiring data for diagnosis of Charcot-Marie-Tooth disease comprising:

determining the nucleotide sequence of MME gene in a sample; and

comparing the sequence with the nucleotide sequence represented by SEQ ID NO: 1 to determine the presence or absence of a mutation.

As described above, mutation in MME gene includes a mutation on any exon or a mutation at a splicing site which inhibits expression of the functional MME protein, and is not limited to any specific mutations. Accordingly, although not limited to any mutations, examples of mutations in MME gene found by the present inventors include a mutation at a splice donor site between exons 7 and 8 (c.654+1G>A) (a mutation of guanine to adenine at position 37341 of SEQ ID NO: 1), a nonsense mutation on exon 8 (c.661C>T, p.Q221X) (a mutation of cytosine to thymine at position 39106 of SEQ ID NO: 1, a mutation of a glutamine residue to a termination codon at position 221 of SEQ ID NO: 2), and a missense mutation on exon 19 (c.1861T>C, p.C621R) (a mutation of thymine to cytosine at position 88926 of SEQ ID NO: 1, a mutation of a cysteine residue to an arginine residue at position 621 of SEQ ID NO: 2).

Cognitive decline is not found in any of cases with MME mutation identified by the present inventors in the present research, and they are very similar in phenotype in that the age of onset was in the late stage and axonal motor sensory neuropathy was presented. Although the function of NEP in the peripheral nervous system has not yet been revealed, it is expected that the mechanism for processing abnormal proteins operates abnormally also in the peripheral nervous system, which may result in axonal degeneration in peripheral nerves.

### <Mutation in FAT3 Gene>

The FAT3 gene, which is located in the long arm of chromosome 11 (11q14.3) and a member of the human FAT gene family, is a gene having high homology to FAT1 and FAT2. The protein encoded by the FAT3 gene is a macromolecule which has an EGF-like motif and a cadherin motif and belongs to the cadherin superfamily as cell adhesion molecules (Tanoue, T. & Takeichi, M., J Cell Sci 118, 2347-53 (2005)). Expression of the Fat3 protein and mRNA has been found in embryonic stem cells, primitive neuroectoderm, fetal brain, infant brain, adult nervous tissue, and prostate, and the Fat3 protein is expected to have an important role in formation and adjustment of an axon bundle in an extracellular matrix surrounding axons in the embryogenesis stage, and has been found to cause dendrites of retinal cells to have an abnormal form in a FAT3 knockout mouse.

The nucleotide sequence of the gene encoding the functional FAT3 protein and the amino acid sequence of the FAT3 protein are represented by SEQ ID NOs: 3 and 4, respectively. The positions of exons in the FAT3 gene sequence represented by SEQ ID NO: 3 are listed in the following Table 2.

TABLE 2
Information on exons in FAT3 gene sequence

| Exon | Length (nucleotides) | Position in SEQ ID NO: 3 |
|---|---|---|
| 1*¹ | 3,309 | 37,817 to 41,125 |
| 2 | 315 | 210,355 to 210,669 |
| 3 | 62 | 383,105 to 383,166 |
| 4 | 315 | 447,577 to 447,891 |
| 5 | 211 | 450,600 to 450,810 |
| 6 | 140 | 459,762 to 459,901 |
| 7 | 276 | 475,664 to 475,939 |
| 8 | 211 | 478,488 to 478,698 |
| 9 | 4,074 | 483,557 to 487,630 |
| 10 | 197 | 490,874 to 491,070 |
| 11 | 154 | 492,083 to 492,236 |
| 12 | 234 | 495,564 to 495,797 |
| 13 | 390 | 517,343 to 517,732 |
| 14 | 215 | 520,591 to 520,805 |
| 15 | 138 | 522,287 to 522,424 |
| 16 | 144 | 523,384 to 523,527 |
| 17 | 198 | 526,283 to 526,480 |
| 18 | 799 | 529,655 to 530,453 |
| 19 | 135 | 542,935 to 543,069 |
| 20 | 158 | 544,886 to 545,043 |
| 21 | 469 | 552,462 to 552,930 |
| 22 | 154 | 566,452 to 566,605 |
| 23 | 656 | 568,459 to 569,114 |
| 24 | 114 | 572,721 to 572,834 |
| 25 | 60 | 574,910 to 574,969 |
| 26 | 36 | 575,577 to 575,612 |
| 27*² | 5,962 | 576,212 to 582,173 |

*¹including 5′UTR: 37,817 to 37,833
*²including 3′UTR: 576,835 to 582,173

If the functional FAT3 protein is not expressed due to mutation in the FAT3 gene, the onset of CMT is expected to occur. Accordingly, one aspect of the present invention is a method for acquiring data for diagnosis of Charcot-Marie-Tooth disease comprising:

determining the nucleotide sequence of FAT3 gene in a sample; and

comparing the sequence with the nucleotide sequence represented by SEQ ID NO: 3 to determine the presence or absence of a mutation.

As described above, mutation in FAT3 gene includes a mutation on any exon or a mutation at a splicing site which inhibits expression of the functional FAT3 protein, and is not limited to any specific mutations. Accordingly, although not limited to any mutations, examples of mutations found by the present inventors include a missense mutation on exon 9 in FAT3 gene (c.6122C>A, p.P2041H) (a mutation of cytosine to adenine at position 484856 of SEQ ID NO: 3, a mutation of a proline residue to a histidine residue at position 2041 of SEQ ID NO: 4) and a missense mutation on exon 18 in FAT3 gene (c.11327G>A, p.C3776Y) (a mutation of guanine to adenine at position 530415 of SEQ ID NO: 3, a mutation of a cysteine residue to a tyrosine residue at position 3776 of SEQ ID NO: 4).

Two cases with FAT3 mutation are different in age of onset. However, severe muscle weakness of the distal lower limb muscle is found in both cases, and the two cases share common, specific central nervous system symptoms (swallowing disorder and tongue atrophy). Although the presence or absence of expression of FAT3 protein in the peripheral nervous system is unknown, FAT3 protein may be involved in the differentiation and maintenance of peripheral nerve cells, and mutation in FAT3 protein is considered to cause some disorder of axon formation, neurite elongation, etc., in the peripheral nervous system.

### <Mutation in SELRC1 Gene>

The SELRC1 gene is located in the short arm of chromosome 1 (1p32.3) and encodes a sell repeat-containing protein. However, the function is completely unknown. Two cases with SELRC1 mutation are those of juvenile-onset axonal motor sensory neuropathy, and share common central nervous system symptoms (cerebellar ataxia) and MRI findings (cerebellar atrophy) and are characterized in a phenotype similar to spinocerebellar ataxia with axonal neuropathy (SCAN1).

The nucleotide sequence of the gene encoding the functional SELRC1 protein and the amino acid sequence of the SELRC1 protein are represented by SEQ ID NOs: 5 and 6, respectively. The positions of exons in the SELRC1 gene sequence represented by SEQ ID NO: 5 are listed in the following Table 3.

TABLE 3
Information on exons in SELRC1 gene sequence

| Exon | Length (nucleotides) | Position in SEQ ID NO: 5 |
|---|---|---|
| 1*¹ | 146 | 1 to 146 |
| 2 | 141 | 5,500 to 5640 |
| 3*² | 1,333 | 10,199 to 11,531 |

*¹including 5′UTR: 1 to 40
*²including 3′UTR: 10,648 to 11,531

If the functional SELRC1 protein is not expressed due to mutation in the SELRC1 gene, the onset of CMT is expected to occur. Accordingly, one aspect of the present invention is a method for acquiring data for diagnosis of Charcot-Marie-Tooth disease comprising:

determining the nucleotide sequence of the SELRC1 gene in a sample; and

comparing the sequence with the nucleotide sequence represented by SEQ ID NO: 5 to determine the presence or absence of a mutation.

As described above, mutation in the SELRC1 gene includes a mutation on any exon or a mutation at a splicing site which inhibits expression of the functional SELRC1 protein, and is not limited to any specific mutations. Accordingly, although not limited to any mutations, examples of mutations found by the present inventors include a missense mutation on exon 2 in the SELRC1 gene (c.115C>T, p.R39W) (a mutation of cytosine to thymine at position 5508 of SEQ ID NO: 5, a mutation of an arginine residue to a tryptophan residue at position 39 of SEQ ID NO: 6) and a missense mutation on exon 1 in the SELRC1 gene (c.17A>G, p.D6G) (a mutation of adenine to guanine at position 57 of SEQ ID NO: 5, a mutation of an aspartic acid residue to a glycine residue at position 6 of SEQ ID NO: 6).

### <Diagnosis of CMT>

The present invention also provides a method for detection of a mutation in a CMT-causing gene and diagnosis of CMT. In the present specification, “detection” occasionally refers to “detection of a mutation” and “detection of a disease or the possibility of the onset of a disease”, and “diagnosis” occasionally encompasses not only “diagnosis” to determine whether a subject is presenting with “CMT” but also “diagnosis” to determine “the possibility of the onset of CMT”.

In one embodiment, the present invention provides a method for diagnosing CMT in a subject comprising:

detecting the presence of a mutation in MME gene, FAT3 gene, or SELRC1 gene in a biological sample; and

diagnosing CMT in the subject on the basis of the presence of a mutation.

In one aspect, the method comprises:

hybridizing a nucleic acid derived from MME gene, FAT3 gene, or SELRC1 gene in a biological sample with at least one oligonucleotide which specifically binds to a mutated MME gene, FAT3 gene, or SELRC1 gene and does not bind to the nonmutated MME gene, FAT3 gene, or SELRC1 gene; and detecting a signal generated through the hybridization.

Detection of a signal can be performed by using a conventional method in the art.

In another aspect, the method comprises:

amplifying a nucleic acid derived from MME gene, FAT3 gene, or SELRC1 gene in a biological sample by using at least one oligonucleotide which specifically binds to a mutated MME gene, FAT3 gene, or SELRC1 gene and does not bind to the nonmutated MME gene, FAT3 gene, or SELRC1 gene; and

detecting the amplified product.

Detection of an amplified product can be performed by using a conventional method in the art.

In another embodiment, the present invention also provides a method for identifying a subject having a high risk of the onset of CMT comprising the above steps.

The above “oligonucleotide” may comprise, but is not limited to, a primer and a probe, and a DNA chip to be described later which have a nucleotide sequence length within a range typically used in the art.

The above “mutation” in particular refers to mutation which does not allow for expression of a functional protein, and specific examples thereof include, but are not limited to, mutations described later. The “nonmutated” MME gene, FAT3 gene, and SELRC1 gene refer to genes having the sequence represented by SEQ ID NOs: 1, 3, and 5, respectively.

Examples of methods for detecting a mutation include: a method in which a primer having a length of 20 to 25 nucleotides is produced on the basis of the sequence of each of the above three genes (SEQ ID NOs: 1, 3, and 5), a nucleic acid in a sample is amplified by PCR method, and then the nucleotide sequence of the amplified nucleic acid is determined; a method in which a probe having a length of about 25 nucleotides and containing a possibly mutated nucleotide site is produced, and hybridization is performed for a DNA chip with the probe immobilized thereon; and PCR-SSCP method, and those skilled in the art could detect a mutation in a gene based on the description of the present specification. In the case that a partial nucleic acid is used to detect a mutation, the sequence to be selected is not limited and may be any partial sequence in the sequence represented by SEQ ID NO: 1, 3, or 5. Those skilled in the art could appropriately determine the sequence of an oligonucleotide which can be used for hybridization with and amplification of a nucleic acid having a specific mutation and synthesize an oligonucleotide suitable for detection of a mutation of interest. The present invention can provide such a primer, probe, and DNA chip.

In addition to the above-mentioned Sanger method and microarray method, which have been conventionally used in the art, a massive parallel nucleotide sequencing method by utilizing a next-generation sequencer (DNA analyzer) can be used for detection of mutations. Detection of mutations using the next-generation sequencer (NGS) includes Target Resequencing, exome analysis, and whole genome sequencing. Target Resequencing, an approach in which a specific sequence containing a targeted gene coding region is captured and concentrated using a special probe or primer and then subjected to sequence analysis, enables detection of a mutation targeting MME gene, FAT3 gene, SELRC1 gene, etc. Exome analysis and whole genome sequencing can determine almost all of the nucleotide sequence of a targeted gene, and thus enable easy detection of a mutation.

For example, in order to detect a mutation actually found in Examples below, in the case of MME gene, a primer or probe containing such a nucleotide site that a mutation of 654+1G>A, 661C>T, or 1861T>C (a mutation of guanine to adenine at position 37341 of SEQ ID NO: 1, a mutation of cytosine to thymine at position 39106 of SEQ ID NO: 1, or a mutation of thymine to cytosine at position 88926 of SEQ ID NO: 1) can be detected is suitably produced.

In the case of FAT3 gene, a primer or probe is suitably produced so as to detect a mutation of 6122C>A or 11327G>A (a mutation of cytosine to adenine at position 484856 of SEQ ID NO: 3 or a mutation of guanine to adenine at position 530415 of SEQ ID NO: 3).

In the case of SELRC1 gene, a primer or probe is suitably produced so as to detect a mutation of 115C>T or 17A>G (a mutation of cytosine to thymine at position 5508 of SEQ ID NO: 5 or a mutation of adenine to guanine at position 57 of SEQ ID NO: 5).

More specifically, a primer or probe having a nucleotide sequence containing, for example, each of the sequences represented by SEQ ID NOs: 17 to 20 is produced.

In the case of detection of a mutation in an mRNA, an in situ hybridization method can be used, for example.

In the case of detection of a mutation in a protein, an immunoassay method can be used, for example, in which the presence or absence of a mutation is determined with an antibody specific to a nonmutated protein, utilizing difference in binding affinity to the antibody.

For example, in order to detect a mutation actually found in Examples below, in the case of MME gene, an assay system is suitably designed so as to detect a mutation of Q221X or C621R (a mutation of a glutamine residue to a termination codon at position 221 of SEQ ID NO: 2 or a mutation of a cysteine residue to an arginine residue at position 621 of SEQ ID NO: 2).

In the case of FAT3 gene, an assay system is suitably designed so as to detect a mutation of P2041H or C3776Y (a mutation of a proline residue to a histidine residue at position 2041 of SEQ ID NO: 4 or a mutation of a cysteine residue to a tyrosine residue at position 3776 of SEQ ID NO: 4).

In the case of SELRC1 gene, an assay system is suitably designed so as to detect a mutation of R39W or D6G (a mutation of an arginine residue to a tryptophan residue at position 39 of SEQ ID NO: 6 or a mutation of an aspartic acid residue to a glycine residue at position 6 of SEQ ID NO: 6).

The present invention also provides a primer or probe for detection of autosomal recessive inherited CMT, being a nucleic acid consisting of a nucleotide sequence represented by any of SEQ ID NOs: 1, 3, and 5 or a partial nucleic acid thereof. The partial nucleic acid is a nucleic acid having a nucleotide sequence of 20 to 25 consecutive nucleotides. Use of the primer or probe according to the present invention enables determination of the presence or absence of a mutation in a DNA in a biological sample derived from a subject.

The present invention also provides a DNA chip for detection of autosomal recessive inherited CMT, comprising the above probe. The DNA chip can be produced by providing each probe singly or in combination with known genes on, for example, a GeneChip® CustomSeq® Resequencing Array manufactured by Affymetrix, although the production method is not limited thereto. Those skilled in the art could easily understand and conduct a procedure for detection of a mutation in a DNA with the above probe or DNA chip.

The present invention also provides a kit for detection of a mutation in MME gene, FAT3 gene, and/or SELRC1 gene in a biological sample to be used for the method according to the present invention. The kit according to the present invention comprises the above primer or probe or the above DNA chip, and a reagent, a buffer, etc., for hybridization with DNA derived from a subject.

### <Four Genes Having Homozygous Nonsense Mutation>

The present inventors further found four genes having a homozygous nonsense mutation as candidate genes for AR-CMT: ABCC3 (ATP-binding cassette, subfamily C (CFTR/MRP), member 3), ANKRD7 (ankyrin repeat domain 7), CNGA4 (cyclic nucleotide-gated channel α4), and COL6A6 (collagen, type VI, α6). The nucleotide sequence and amino acid sequence for ABCC3, ANKRD7, CNGA4, and COL6A6 are represented by SEQ ID NOs 7 and 8, SEQ ID NOs 9 and 10, SEQ ID NOs 11 and 12, and SEQ ID NOs 13 and 14, respectively. The positions of exons in these nucleotide sequences are listed in Tables 4 to 7.

TABLE 4
Information on exons in ABCC3 gene sequence

| Exon | Length (nucleotides) | Position in SEQ ID NO: 7 |
|---|---|---|
| 1*¹ | 125 | 1 to 125 |
| 2 | 177 | 20,976 to 21,152 |
| 3 | 126 | 21,846 to 21,971 |
| 4 | 138 | 22,190 to 22,327 |
| 5 | 126 | 23,226 to 23,351 |
| 6 | 62 | 23,579 to 23,640 |
| 7 | 132 | 24,381 to 24,512 |
| 8 | 192 | 26,067 to 26,258 |
| 9 | 178 | 28,825 to 29,002 |
| 10 | 162 | 29,094 to 29,255 |
| 11 | 93 | 30,297 to 30,389 |
| 12 | 204 | 32,698 to 32,901 |
| 13 | 147 | 33,007 to 33,153 |
| 14 | 88 | 33,574 to 33,661 |
| 15 | 67 | 33,997 to 34,063 |
| 16 | 127 | 34,284 to 34,410 |
| 17 | 177 | 34,496 to 34,672 |
| 18 | 168 | 38,115 to 38,282 |
| 19 | 190 | 38,613 to 38,802 |
| 20 | 115 | 40,506 to 40,620 |
| 21 | 145 | 40,775 to 40,919 |
| 22 | 208 | 41,027 to 41,234 |
| 23 | 311 | 41,422 to 41,732 |
| 24 | 200 | 42,888 to 43,087 |
| 25 | 127 | 43,237 to 43,363 |
| 26 | 102 | 44,942 to 45,043 |
| 27 | 147 | 48,754 to 48,900 |
| 28 | 159 | 49,093 to 49,251 |
| 29 | 167 | 49,853 to 50,019 |
| 30 | 195 | 52,680 to 52,874 |
| 31*² | 1161 | 56,236 to 57,396 |

*¹including 5′UTR: 1 to 80
*²including 3′UTR: 56,345 to 57,396

TABLE 5
Information on exons in ANKRD7 gene sequence

| Exon | Length (nucleotides) | Position in SEQ ID NO: 9 |
|---|---|---|
| 1*¹ | 334 | 238 to 571 |
| 2 | 115 | 9,993 to 10,107 |
| 3 | 174 | 10,263 to 10,436 |
| 4 | 107 | 11,603 to 11,709 |
| 5 | 137 | 12,352 to 12,488 |
| 6*² | 90 | 15,471 to 15,560 |
| 7*² | 341 | 17,911 to 18,251 |

*¹including 5′UTR: 238 to 392
*²including 3′UTR: 15,525 to 18,251

TABLE 6
Information on exons in CNGA4 gene sequence

| Exon | Length (nucleotides) | Position in SEQ ID NO: 11 |
|---|---|---|
| 1*¹ | 177 | 3,599 to 3,775 |
| 2 | 102 | 3,891 to 3,992 |
| 3 | 107 | 4,191 to 4,297 |
| 4 | 646 | 4,573 to 5,218 |
| 5 | 350 | 5,938 to 6,287 |
| 6*² | 481 | 8,456 to 8,936 |

*¹including 5′UTR: 3,599 to 3,713
*²including 3′UTR: 8,917 to 8,936

TABLE 7
Information on exons in COL6A6 gene sequence

| Exon | Length (nucleotides) | Position in SEQ ID NO: 13 |
|---|---|---|
| 1*¹ | 95 | 1 to 95 |
| 2 | 597 | 2,735 to 3,331 |
| 3 | 621 | 4,661 to 5,281 |
| 4 | 561 | 6,369 to 6,929 |
| 5 | 558 | 7,714 to 8,271 |
| 6 | 576 | 10,485 to 11,060 |
| 7 | 570 | 13,623 to 14,192 |
| 8 | 344 | 21,228 to 21,571 |
| 9 | 79 | 21,657 to 21,735 |
| 10 | 155 | 26,173 to 26,327 |
| 11 | 93 | 28,757 to 28,849 |
| 12 | 54 | 30,708 to 30,761 |
| 13 | 72 | 32,208 to 32,279 |
| 14 | 27 | 32,363 to 32,389 |
| 15 | 45 | 32,728 to 32,772 |
| 16 | 54 | 32,866 to 32,919 |
| 17 | 63 | 33,948 to 34,010 |
| 18 | 66 | 38,032 to 38,097 |
| 19 | 54 | 39,424 to 39,477 |
| 20 | 36 | 46,598 to 46,633 |
| 21 | 63 | 48,569 to 48,631 |
| 22 | 63 | 50,316 to 50,378 |
| 23 | 63 | 61,488 to 61,550 |
| 24 | 63 | 66,152 to 66,214 |
| 25 | 51 | 66,986 to 67,036 |
| 26 | 36 | 74,257 to 74,292 |
| 27 | 63 | 75,366 to 75,428 |
| 28 | 63 | 81,319 to 81,381 |
| 29 | 36 | 82,499 to 82,534 |
| 30 | 37 | 82,654 to 82,690 |
| 31 | 12 | 84,658 to 84,669 |
| 32 | 494 | 88,736 to 89,229 |
| 33 | 97 | 98,343 to 98,439 |
| 34 | 672 | 101,304 to 101,975 |
| 35 | 94 | 104,670 to 104,763 |
| 36*¹ | 2,954 | 114,869 to 117,822 |

*¹including 5′UTR: 1 to 31
*²including 3′UTR: 115,065 to 117,822

The protein encoded by ABCC3 gene, which is located on chromosome 17, belongs to the ABC (ATP-binding cassette) transporter superfamily, and is involved in efflux transport from the inside of a cell in collaboration with ATP hydrolysis. In particular, the protein is known to play an important role for transport and excretion of glucuronic acid conjugates, organic anionic compounds, and bile acid in liver and intestinal tract.

According to a recent report on a genome-wide association study (GWAS) for a habitual alcohol drinker group, ANKRD7 gene located on chromosome 7 is a gene associated with the risk of alcoholism. However, the function has not been clarified.

CNGA4 gene located on chromosome 11 is known to encode a regulatory subunit for a cyclic nucleotide-gated channel and have an important role for olfactory signaling and adaptation in olfactory neurons.

COL6A6 gene located on chromosome 3 is a gene encoding α6 chain of type VI collagen, an extracellular matrix protein. Mutations in COL6A1, COL6A2, and COL6A3 are known to cause Ullrich congenital muscular dystrophy (OMIM #254090) and Bethlem myopathy (OMIM#158810). However, influence of COL6A6 mutation on human phenotype has not been reported. The type VI collagen has been reported to have a neuroprotective action to prevent the aggregation of amyloid β proteins, which causes Alzheimer's disease.

A mutation found in ABCC3 gene is a homozygous nonsense mutation on exon 30 (48764928 C>T R1438X) (a mutation of cytosine to thymine at position 52711 of SEQ ID NO: 7, a mutation of an arginine residue to a termination codon at position 1438 of SEQ ID NO: 8).

A mutation found in ANKRD7 gene is a homozygous nonsense mutation (117874773 G>T E105X) (a mutation of guanine to thymine at position 10281 of SEQ ID NO: 9, a mutation of a glutamic acid residue to a termination codon at position 105 of SEQ ID NO: 10).

A mutation found in CNGA4 gene is a homozygous nonsense mutation (6261928 C>T R302X) (a mutation of cytosine to thymine at position 5205 of SEQ ID NO: 11, a mutation of an arginine residue to a termination codon at position 302 of SEQ ID NO: 12).

A mutation found in COL6A6 gene is a homozygous nonsense mutation (130282181 C>T Q112X) (a mutation of cytosine to thymine at position 3004 of SEQ ID NO: 13, a mutation of a glutamine residue to a termination codon at position 112 of SEQ ID NO: 14).

Accordingly, detection of a mutation in ABCC3 gene, ANKRD7 gene, CNGA4 gene, and/or COL6A6 gene enables acquisition of data for diagnosis of AR-CMT.

Detection of a mutation is not limited, and detection of a mutation in ABCC3 gene involves, for example, detection of a mutation in a gene on chromosome 17 such as 48764928 C>T R1438X.

Detection of a mutation in ANKRD7 gene involves detection of a mutation in a gene on chromosome 7 such as 117874773 G>T E105X.

Detection of a mutation in CNGA4 gene involves detection of a mutation in a gene on chromosome 11 such as 6261928 C>T R302X.

Detection of a mutation in COL6A6 gene involves detection of a mutation in a gene on chromosome 3 such as 130282181 C>T Q112X.

The present invention also provides a method for diagnosing AR-CMT in a subject comprising:

detecting the presence of a mutation in ABCC3 gene, ANKRD7 gene, CNGA4 gene, and/or COL6A6 gene in a biological sample; and

diagnosing AR-CMT in the subject on the basis of the presence of a mutation.

In one aspect, the method comprises:

hybridizing a nucleic acid derived from ABCC3 gene, ANKRD7 gene, CNGA4 gene, or COL6A6 gene in a biological sample with at least one oligonucleotide which specifically binds to a mutated ABCC3 gene, ANKRD7 gene, CNGA4 gene, or COL6A6 gene and does not bind to the nonmutated ABCC3 gene, ANKRD7 gene, CNGA4 gene, or COL6A6 gene; and

detecting a signal generated through the hybridization.

In another aspect, the method comprises:

amplifying a nucleic acid derived from ABCC3 gene, ANKRD7 gene, CNGA4 gene, or COL6A6 gene in a biological sample by using at least one oligonucleotide which specifically binds to a mutated ABCC3 gene, ANKRD7 gene, CNGA4 gene, or COL6A6 gene and does not bind to the nonmutated ABCC3 gene, ANKRD7 gene, CNGA4 gene, or COL6A6 gene; and

detecting the amplified product.

In another embodiment, the present invention also provides a method for identifying a subject having a high risk of the onset of AR-CMT comprising the above steps.

The above “mutation” in particular refers to mutation which does not allow for expression of a functional protein, and specific examples thereof include, but are not limited to, the mutations described above. The “nonmutated” ABCC3 gene, ANKRD7 gene, CNGA4 gene, and COL6A6 gene refer to genes having a sequence represented by SEQ ID NOs: 7, 9, 11, and 13, respectively.

In addition, the present invention can provide: a primer or probe having a nucleic acid consisting of a nucleotide sequence of any of the above four genes represented by SEQ ID NOs: 7, 9, 11, and 13, or a partial nucleic acid thereof; a DNA chip comprising it; and a kit comprising at least one of them. Those skilled in the art could appropriately determine the sequence of an oligonucleotide which can be used for hybridization with and amplification of a nucleic acid having a specific mutation and synthesize an oligonucleotide suitable for detection of a mutation of interest. The present invention can provide such a primer, probe, and DNA chip.

### <Mutation in CAD Gene>

The present inventors have further found cases in each of which the translated region of CAD (carbamoyl-phosphate synthetase 2, aspartate transcarbamylase, and dihydroorotase) gene has two heterozygous missense mutations. A protein encoded by CAD is a multienzyme complex having the first three enzymatic functions in the de novo biosynthesis pathway of pyrimidine nucleotide (C: carbamoyl-phosphate synthetase (CPS II), A: aspartate carbamoyltransferase, D: dihydroorotase). Although the function of CAD in the peripheral nervous system is unclear, CAD is known to be present in the cytoplasm and closely associated with cellular proliferative potential, and to have high activity in normal cells with active division and proliferation in thymus, testis, spleen, etc., and in various tumor cells.

The nucleotide sequence of the gene encoding the functional CAD protein and the amino acid sequence of the CAD protein are represented by SEQ ID NOs: 15 and 16, respectively. The positions of exons in the CAD gene sequence represented by SEQ ID NO: 15 are listed in the following Table 8.

TABLE 8
Information on exons in CAD gene sequence

| Exon | Length (nucleotides) | Position in SEQ ID NO: 15 |
|---|---|---|
| 1*¹ | 244 | 1 to 244 |
| 2 | 140 | 488 to 627 |
| 3 | 130 | 3,829 to 3,958 |
| 4 | 143 | 4,805 to 4,947 |
| 5 | 142 | 5,130 to 5,271 |
| 6 | 172 | 5,477 to 5,648 |
| 7 | 186 | 6,174 to 6,359 |
| 8 | 113 | 6,528 to 6,640 |
| 9 | 146 | 6,956 to 7,101 |
| 10 | 132 | 7,356 to 7,487 |
| 11 | 234 | 7,621 to 7,854 |
| 12 | 222 | 8,316 to 8,537 |
| 13 | 189 | 8,742 to 8,930 |
| 14 | 125 | 9,136 to 9,260 |
| 15 | 131 | 9,443 to 9,573 |
| 16 | 113 | 14,079 to 14,191 |
| 17 | 245 | 14,591 to 14,835 |
| 18 | 247 | 15,059 to 15,305 |
| 19 | 99 | 15,653 to 15,751 |
| 20 | 225 | 15,923 to 16,147 |
| 21 | 183 | 16,237 to 16,419 |
| 22 | 219 | 16,619 to 16,837 |
| 23 | 168 | 17,129 to 17,296 |
| 24 | 183 | 17,856 to 18,038 |
| 25 | 105 | 18,147 to 18,251 |
| 26 | 240 | 18,895 to 19,134 |
| 27 | 82 | 19,360 to 19,441 |
| 28 | 167 | 19,990 to 20,156 |
| 29 | 165 | 20,329 to 20,493 |
| 30 | 132 | 20,667 to 20,798 |
| 31 | 202 | 21,042 to 21,243 |
| 32 | 191 | 21,653 to 21,843 |
| 33 | 141 | 21,942 to 22,082 |
| 34 | 102 | 22,308 to 22,409 |
| 35 | 97 | 22,876 to 22,972 |
| 36 | 45 | 23,522 to 23,566 |
| 37 | 170 | 23,669 to 23,838 |
| 38 | 75 | 23,933 to 24,007 |
| 39 | 213 | 24,522 to 24,734 |
| 40 | 126 | 24,913 to 25,038 |
| 41 | 156 | 25,231 to 25,386 |
| 42 | 102 | 25,483 to 25,584 |
| 43 | 95 | 25,809 to 25,903 |
| 44*² | 528 | 26,027 to 26,554 |

*¹including 5′UTR: 1 to 162
*²including 3′UTR: 26,130 to 26,554

As described above, mutation in CAD gene includes a mutation on any exon or a mutation at a splicing site which inhibits expression of the functional CAD protein, and is not limited to any specific mutations. Accordingly, although not limited to any mutations, examples of mutations in CAD gene found by the present inventors include heterozygous mutations of c.497C>T, p.T166I (a mutation of cytosine to thymine at position 5131 of SEQ ID NO: 15, a mutation of a threonine residue to an isoleucine residue at position 166 of SEQ ID NO: 16) and c.503G>A, p.R168Q (a mutation of guanine to adenine at position 5137 of SEQ ID NO: 15, a mutation of an arginine residue to a glutamine residue at position 168 of SEQ ID NO: 16) and heterozygous mutations of c.2501G>A, p.R834H (a mutation of guanine to adenine at position 14691 of SEQ ID NO: 15, a mutation of an arginine residue to a histidine residue at position 834 of SEQ ID NO: 16) and c.4958T>G, p.L1653R (a mutation of thymine to guanine at position 21139 of SEQ ID NO: 15, a mutation of a leucine residue to an arginine residue at position 1653 of SEQ ID NO: 16).

Accordingly, detection of a mutation in CAD gene enables acquisition of data for diagnosis of CMT. Detection of a mutation is not limited, and involves, for example, detection of one or more mutations of the above c.497C>T, p.T166I, c.503G>A, p.R168Q, c.2501G>A, p.R834H, and c.4958T>G, p.L1653R.

The present invention also provides a method for diagnosing CMT in a subject comprising:

detecting the presence of a mutation in CAD gene in a biological sample; and

diagnosing CMT in the subject on the basis of the presence of a mutation.

In one aspect, the method comprises:

hybridizing a nucleic acid derived from CAD gene in a biological sample with at least one oligonucleotide which specifically binds to a mutated CAD gene and does not bind to the nonmutated CAD gene; and

detecting a signal generated through the hybridization.

In another aspect, the method comprises:

amplifying a nucleic acid derived from CAD gene in a biological sample by using at least one oligonucleotide which specifically binds to a mutated CAD gene and does not bind to the nonmutated CAD gene; and

detecting the amplified product.

In another embodiment, the present invention also provides a method for identifying a subject having a high risk of the onset of CMT comprising the above steps.

The above “mutation” in particular refers to mutation which does not allow for expression of a functional protein, and specific examples thereof include, but are not limited to, the mutations described above. The “nonmutated” CAD gene refers to a gene having a sequence represented by SEQ ID NO: 15.

In addition, the present invention can provide: a primer or probe having a nucleic acid consisting of a nucleotide sequence of the CAD gene represented by SEQ ID NO: 15, or a partial nucleic acid thereof; a DNA chip comprising it; and a kit comprising at least one of them. Those skilled in the art could appropriately determine the sequence of an oligonucleotide which can be used for hybridization with and amplification of a nucleic acid having a specific mutation and synthesize an oligonucleotide suitable for detection of a mutation of interest. The present invention can provide such a primer, probe, and DNA chip.

Moreover, the present invention can provide a method for gene therapy to introduce MME (membrane metallo-endopeptidase) gene, FAT3 (FAT tumor suppressor homolog 3) gene, and/or SELRC1 (Sell repeat containing 1) gene into a CMT patient who has been found to have a mutation in these genes by using the method according to the present invention, or for treatment, for example, to alleviate the symptoms of CMT in a CMT patient by administration of a protein encoded by these genes.

From the result that five unrelated families shared a mutation in MME gene and were presenting with an identical phenotype, the present inventors have found that the gene mutation causes CMT. The types of MME mutations include homozygous nonsense mutation in one case and splicing site mutation in three cases, and thus functional loss of the gene is considered to be highly associated with the pathological mechanism. Also for FAT3 gene and SELRC1 gene which cause the symptoms of CMT due to homozygous missense mutation and ABCC3 gene, ANKRD7 gene, CNGA4 gene, and COL6A6 gene which are expected to cause the symptoms of CMT due to homozygous nonsense mutation, their functional loss may be associated with the pathology condition. If a homozygous or compound heterozygous mutation is present in any of these genes, the function of the corresponding gene is lost and thus the onset is expected to occur at an extremely high probability, even in the case of an individual presenting with no symptoms.

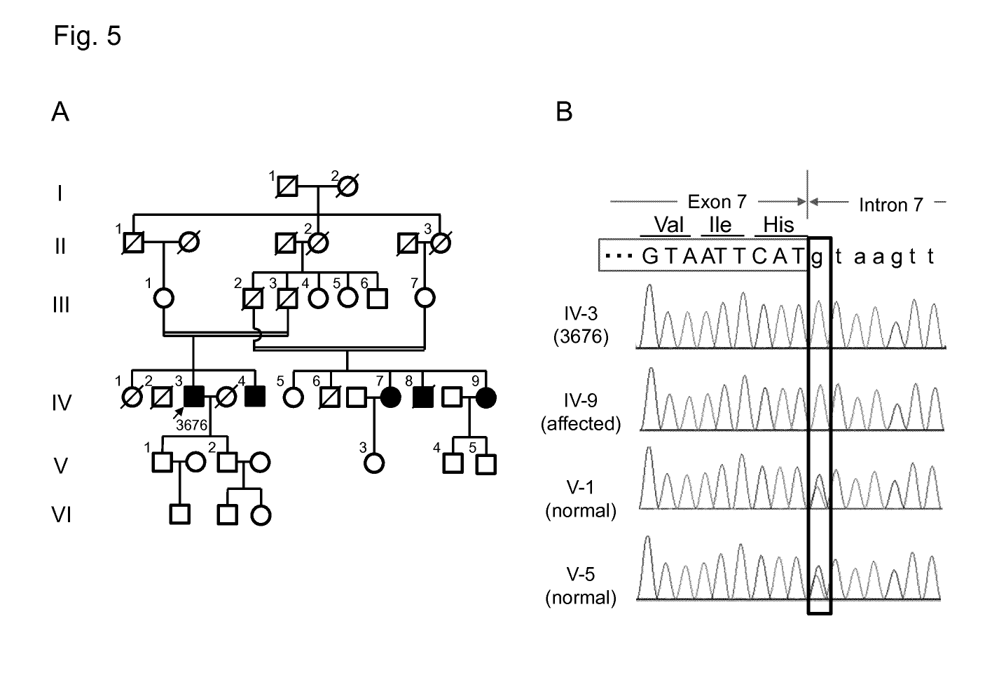

Segregation analysis for one family having an MME gene mutation, two families having a FAT3 gene mutation, and two families having an SELRC1 gene mutation demonstrated that the gene mutations were segregated with the disease in each family, in other words, only affected individuals in the family had a homozygous mutation at the relevant site and healthy individuals in the family had no mutation at the relevant site or had a heterozygous mutation at the relevant site (FIGS. 5 to 8).

Due to the diversity of the disease, all of the above gene mutations are not always found in all patients. However, CMT is an extremely diverse disease genetically and a large number of causal genes therefor are presumably left unknown. As more and more causal genes are discovered, the accuracy of genetic diagnosis is expected to be further improved.

Further, the present inventors have identified many novel potential causal genes and candidate genes for CMT from numerous mutation data in exome analysis for CMT cases using the “disease candidate gene narrowing-down system”. This approach is applicable to identification of cause for a lot of other Mendelian genetic diseases with unidentified cause, and will enable identification of causal genes for more and more diseases further in the future. In addition, identification of novel causal genes for CMT promotes understanding of the molecular pathological mechanism, and is expected to ultimately lead to development of an effective treatment method and understanding of related diseases.

Several approaches have been previously used for discovery of a novel causal gene through exome analysis. The most common approach is one in which, for a family for which causal loci have been narrowed down to a certain level on the basis of the linkage analysis information, a mutation present in the candidate region is found out using exome analysis. In this approach, however, only one causal gene can be found out in most cases, and it is difficult to identify the cause even when exome analysis is performed, if causal loci have not been narrowed down sufficiently in linkage analysis. Linkage analysis requires gene analysis for as many constituent members in a family as possible, including not only a proband but also individuals presenting with symptoms in the family and healthy individuals in the family. In particular, decrease in the number of children and increase in the number of nuclear families are progressing in recent years, and presumably mainly because of this phenomenon, the numbers of families having a small number of family members, families whose family members do not participate in a genetic test, and families with a sporadic case are increasing. As a result, linkage analysis cannot narrow down candidate regions sufficiently, and due to such a situation there still exist many families for which even exome analysis cannot identify the cause.

The present invention is characterized in that only a proband has to be subjected to exome analysis and a plurality of causal genes can be detected or found out simultaneously. However, the number of specimens for exome analysis is still important, and more causal genes are presumably extracted at a higher probability as the number of specimens increases.

### Examples

Hereinafter, the present invention will be described more specifically with reference to Examples, but it is not intended to limit the present invention to the Examples.

### <Patients of Interest and Collection of Clinical Information>

DNAs were collected for 544 cases in total between April 2007 and April 2012, which consisted of cases clinically diagnosed as CMT (including CMT-related diseases) and cases suspected of CMT. Among cases of demyelinating CMT, only cases for which the absence of duplication or deletion in PMP22 gene had been confirmed in advance by using a fluorescence in situ hybridization (FISH) method were collected. Clinical information about patients was acquired by neurologists or pediatricians via medical examinations and tests. The information acquired included clinical course, neurological findings and laboratory findings including blood test, nerve conduction test, and neuroimaging test.

<Screening for Non-Autosomal Dominant Inherited CMT Cases with Unidentified Cause>

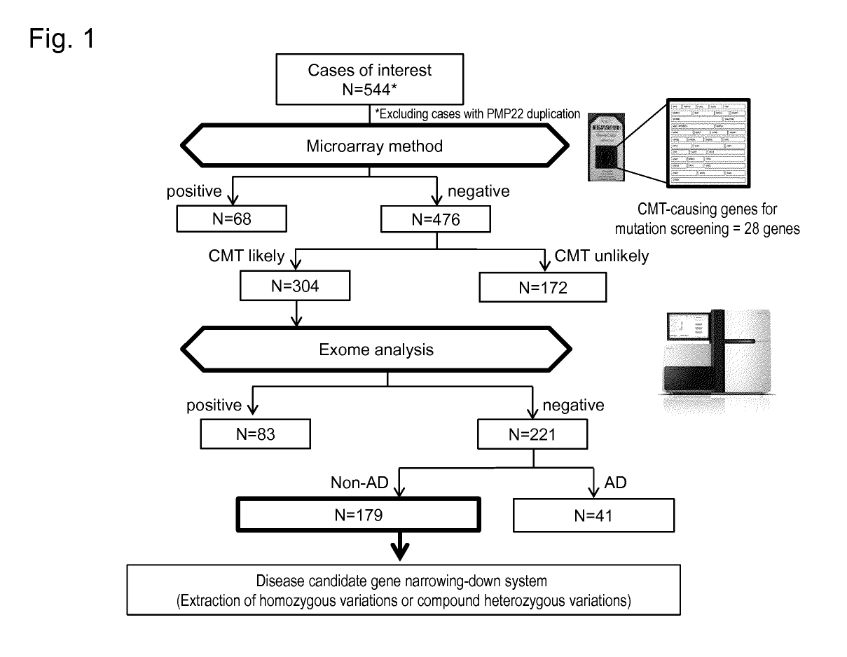

From the 544 cases, 179 cases with no known genetic etiology and a family history of autosomal dominant inheritance were selected according to the following four exclusion criteria (FIG. 1).

(1) Mutation screening with a microarray DNA chip for diagnosis was conducted for all of the 544 cases to exclude 68 cases with pathological mutation in known CMT-causing genes (Table 9) (N=476). (Cases caused by genes being not of interest were excluded.)
(2) From the 476 cases, 172 cases in total including cases with poor clinical information, cases highly suspected of CIDP, and cases with mild neuropathy and main symptoms of other neurological signs (spasticity, rigidity, upper motor neuron sign, etc.) were excluded, and exome analysis was performed for 304 cases. (Cases with the possibility of other diseases were excluded.)
(3) Mutation screening by exome analysis was conducted to exclude 83 cases with known or suspected pathogenic mutation in known causal genes for hereditary neuromuscular diseases which need to be distinguished from CMT and CMT-related diseases such as HMN, HSAN, SMA, and other CMTs (N=221). (Cases caused by genes being not of interest were excluded.)
(4) On the basis of clinical information, 41 cases with a family history of autosomal dominant inherited disease were excluded (N=179).

TABLE 9
The List of screening genes by Microarray resequencing DNA chip

| | Gene | | Phenotype |
|---|---|---|---|
| | symbol | | MIM |
| Classification | (Gene ID) | Phenotype | number |
| CMT | AARS | Charcot-Marie-Tooth disease, axonal, type 2N | 613287 |
| | (16) | | |
| CMT | DHH | 46XY partial gonadal dysgenesis, with minifascicular neuropathy | 607080 |
| | (50846) | | |
| CMT | EGR2 | Charcot-Marie-Tooth disease, type 1D | 607678 |
| | (1959) | | |
| CMT | GAN | Giant axonal neuropathy-1 | 256850 |
| | (8139) | | |
| CMT | GARS | Charcot-Marie-Tooth disease, type 2D | 601472 |
| | (2617) | Neuropathy, distal hereditary motor, type V | 600794 |
| CMT | GDAP1 | Charcot-Marie-Tooth disease, axonal, type 2K | 607831 |
| | (54332) | Charcot-Marie-Tooth disease, axonal, with vocal cord paresis | 607706 |
| | | Charcot-Marie-Tooth disease, recessive intermediate, A | 608340 |
| | | Charcot-Marie-Tooth disease, type 4A | 214400 |
| CMT | GJB1 | Charcot-Marie-Tooth neuropathy, X-linked dominant, 1 | 302800 |
| | (2705) | | |
| CMT | HSPB1 | Charcot-Marie-Tooth disease, axonal, type 2F | 606595 |
| | (3315) | Neuropathy, distal hereditary motor, type IIB | 608634 |
| CMT | HSPB8 | Charcot-Marie-Tooth disease, axonal, type 2L | 608673 |
| | (26353) | Neuropathy, distal hereditary motor, type IIA | 158590 |
| CMT | KARS | Charcot-Marie-Tooth disease, recessive intermediate, B | 613641 |
| | (3735) | | |
| CMT | LITAF | Charcot-Marie-Tooth disease, type 1C | 601098 |
| | (9516) | | |
| CMT | LMNA | Charcot-Marie-Tooth disease, type 2B1 | 605588 |
| | (4000) | | |
| CMT | MFN2 | Charcot-Marie-Tooth disease, type 2A2 | 609260 |
| | (9927) | Hereditary motor and sensory neuropathy VI | 601152 |
| CMT | MPZ | Charcot-Marie-Tooth disease, dominant intermediate D | 607791 |
| | (4359) | Charcot-Marie-Tooth disease, type 1B | 118200 |
| | | Charcot-Marie-Tooth disease, type 2I | 607677 |
| | | Charcot-Marie-Tooth disease, type 2J | 607736 |
| | | Dejerine-Sottas disease | 145900 |
| | | Neuropathy, congenital hypomyelinating | 605253 |
| CMT | MTMR2 | Charcot-Marie-Tooth disease, type 4B1 | 601382 |
| | (8898) | | |
| CMT | NDRG1 | Charcot-Marie-Tooth disease, type 4D | 601455 |
| | (10397) | | |
| CMT | NEFL | Charcot-Marie-Tooth disease, type 1F | 607734 |
| | (4747) | Charcot-Marie-Tooth disease, type 2E | 607684 |
| CMT | PMP22 | Charcot-Marie-Tooth disease, type 1A | 118220 |
| | (5376) | Charcot-Marie-Tooth disease, type 1E | 118300 |
| | | Dejerine-Sottas disease | 145900 |
| | | Neuropathy, recurrent, with pressure palsies | 162500 |
| CMT | PRX | Charcot-Marie-Tooth disease, type 4F | 614895 |
| | (57716) | Dejerine-Sottas disease, autosomal recessive | 145900 |
| CMT | RAB7A | Charcot-Marie-Tooth disease, type 2B | 600882 |
| | (7879) | | |
| CMT | SBF2 | Charcot-Marie-Tooth disease, type 4B2 | 604563 |
| | (81846) | | |
| CMT | SH3TC2 | Charcot-Marie-Tooth disease, type 4C | 601596 |
| | (79628) | | |
| CMT | YARS | Charcot-Marie-Tooth disease, dominant intermediate C | 608323 |
| | (8565) | | |
| CMT, distal | DNM2 | Charcot-Marie-Tooth disease, axonal, type 2M | 606482 |
| myopathies | (1785) | Charcot-Marie-Tooth disease, dominant intermediate B | 606482 |
| Other hereditary | SLC12A6 | Agenesis of the corpus callosum with peripheral neuropathy | 218000 |
| neuropathies | (9990) | | |
| Other inherited disorders | APTX | Ataxia, early-onset, with oculomotor apraxia and | 208920 |
| affecting the peripheral | (54840) | hypoalbuminemia | |
| nervous system | | | |
| Other inherited disorders | TDP1 | Spinocerebellar ataxia, autosomal recessive, | 607250 |
| affecting the peripheral | (55775) | with axonal neuropathy | |
| nervous system | | | |
| ALS, HMN | SETX | Amyotrophic lateral sclerosis 4, juvenile | 602433 |
| | (23064) | Ataxia- ocular apraxia-2 | 606002 |

### <Microarray DNA Chip for Diagnosis>

A DNA chip for diagnosis (GeneChip® CustomSeq® Resequencing Array manufactured by Affymetrix) provided with 28 known causal genes for CMT and CMT-related diseases (Table 9) was uniquely designed and mutation analysis was performed.

Examples of databases providing the gene sequences include RefSeq (Reference Sequence database) available from NCBI (National Center for Biotechnology Information; http://www.ncbi.nlm.nih.gov/gene) and hg19 (GRCh37 or GRCh38) available from UCSC genome database.

“Gene ID” in Table 9 indicates the ID of each gene in the database from NCBI. Gene sequence information in the database can be obtained as follows.

Access to NCBI (http://www.ncbi.nlm.nih.gov/gene)→Input Gene symbol (or Gene ID) in the search box and search→Click “FASTA” in “Genomic” in the item “NCBI Reference Sequences (RefSeq)”→The whole sequence will be displayed.

For each case, 120 ng of DNA was used. A primer set targeting the whole translated regions and slicing sites of genes provided was produced, and the genome DNA was amplified using multiplex PCR, and pooling, fragmentation, and labeling were performed in accordance with the protocol (Affymetrix CustomSeq Resequencing protocol instructions). The chip was subjected to hybridization (49° C., 60 RPM, 16 hours) followed by washing, and thereafter stained with biotin and scanned. The microarray data were analyzed with GeneChip sequence Analysis Software version 4.0 (Affymetrix).

### <Exome Analysis>

Genome DNA was extracted from the peripheral blood of each patient by using Gentra Puregene Blood Kit (QIAGEN, Tokyo, Japan).

By using a SureSelect v4+UTR kit, an exon concentration kit manufactured by Illumina, Inc., 3 μg of genome DNA was subjected to exon capture followed by exome analysis with a Hiseq2000 (Illumina, Inc., San Diego, Calif.). After acquisition of sequence data, the raw read sequence was aligned with a human reference sequence (National Center for Biotechnology Information reference genome build 37/UCSC human genome 19) by using Burrows-Wheeler Alingner (BWA) (see Li, H. & Durbin, R. Fast and accurate short read alignment with Burrows-Wheeler transform. Bioinformatics 25, 1754-60 (2009)), and mutation calling was performed by using SAMtools (see Li, H. et al. The Sequence Alignment/Map format and SAMtools. Bioinformatics 25, 2078-9 (2009)). Annotation was performed with an in-house script.

### <Disease Candidate Gene Narrowing-Down System>

To efficiently identify candidates of causal mutations or causal genes for monogenic diseases from numerous mutations obtained in exome analysis, we developed a “disease candidate gene narrowing-down system”. This system is composed of a “filtering system” and “shared variants pickup system”. The “filtering system” can filter mutations called in the exome analysis for each case on the basis of the class of mutation (SNV (single nucleotide variant) or INDEL (minute insertion/deletion)), mutation type (synonymous mutation, non-synonymous mutation, nonsense mutation, frameshift mutation, splicing site mutation, intron mutation, 5′/3′-UTR mutation), genotype (heterozygous or homozygous), quality, read depth, MAF (minor allele frequency), registration status in public databases, etc. The “shared variants pickup system” is a system to efficiently extract mutations or mutated genes shared by a plurality of affected individuals.

### <Mutation Filtering and Overlap Strategy>

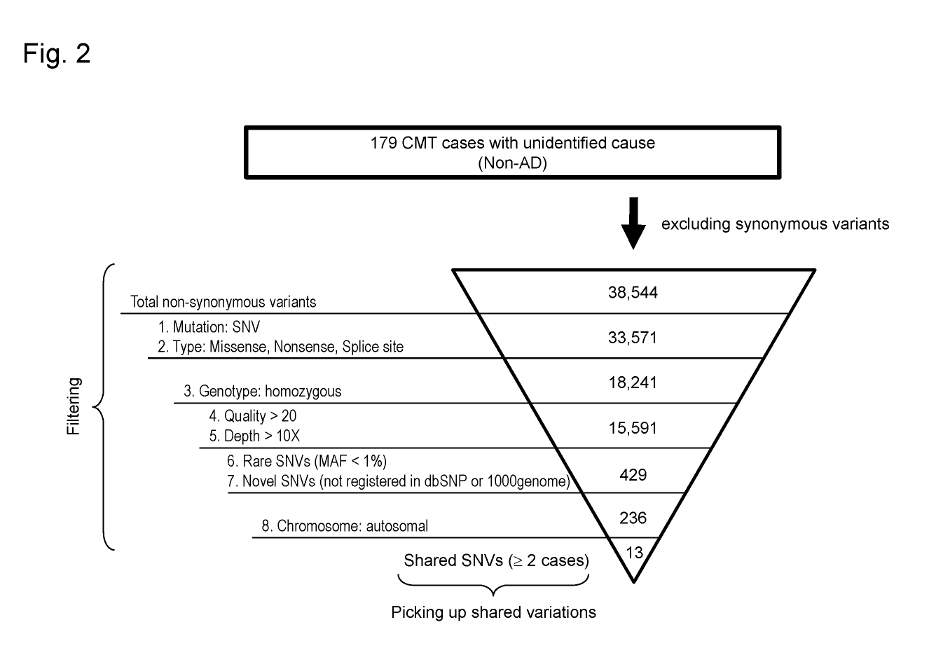

From the exome analysis for the above-described 179 cases of non-autosomal dominant inherited (Non-AD) CMT with unidentified cause, a massive mutation list was obtained. For the purpose of identifying novel causal genes for autosomal recessive inherited CMT, we filtered the mutation list using the “disease candidate gene narrowing-down system” under the following eight conditions (FIG. 2).

(1) Class of mutation: SNV
(2) Mutation type: non-synonymous mutation (non-synonymous SNV), frameshift/in-flame mutation (frameshift/in-frame InDel), splicing site mutation
(3) Genotype: homozygous mutation or compound heterozygous mutation
(4) Quality: Phred scaled base quality >20 and Phred scaled mapping quality >20
(5) Coverage (Read depth)>10×
(6) MAF (minor allele frequency) according to 1000 Genomes Project36<1%
(7) A novel mutation not registered in 1000 genomes database (http://brower.1000genomes.org) or dbSNP database build 137 (http://www.ncbi.nlm.nih.gov/snp/)
(8) Chromosome: autosome (chrom 1 to 22)

After filtering, mutations or mutated genes shared by two or more cases were extracted and listed using the “shared variants pickup system”.

<Comparison with Japanese Control Database>

Mutations not registered in 1000 Genomes (http://www.1000genomes.org/) or dbSNP (build 137) (http://www.ncbi.nlm.nih.gov/projects/SNP/) may include many mutations specific to Japanese. Thus, we referred to the WEB site Human Genetic Variation Browser (http://www.genome.med.kyoto-u.ac.jp/SnpDB/), which accumulates mutation data in exome analysis for approximately 1,000 Japanese control specimens and discloses the data partially, and compared and checked the information on mutation frequency extracted by the “disease candidate gene narrowing-down system”.

### <Mutation Confirmation Using Direct Sequencing Method>

All of the mutations in known causal genes detected by the microarray DNA chip and exome analysis were reconfirmed using Sanger method. In addition, the mutations extracted by using the “disease candidate gene narrowing-down system” were reconfirmed using Sanger method, and segregation analysis was performed as thorough as possible.

### <Extraction of Novel Candidate Genes>

The mutation data in exome analysis for the 179 cases of non-autosomal dominant inherited (Non-AD) CMT with unidentified cause, for which pathological mutation was not found in known causal genes for CMT (and CMT-related diseases), were filtered using the “disease candidate gene narrowing-down system”, and then 19 genes (41 mutations) each sharing a homozygous mutation or a compound heterozygous mutation among two or more cases were found out.

From the 41 mutations, mutations registered in the Japanese control database were excluded, and four novel potential causal genes for CMT, i.e., MME (membrane metallo-endopeptidase) gene, FAT3 (FAT atypical cadherin 3) gene, SELRC1 (Sell repeat containing 1) gene, CAD (carbamoyl-phosphate synthetase 2, aspartate transcarbamylase, and dihydroorotase) gene, were newly found out (Table 10). Clinical features of cases with these gene mutations are summarized in Tables 11 to 14.

TABLE 10
Mutation sites and Prediction scores for novel causal genes

| | | | Amino | | | | | | |
|---|---|---|---|---|---|---|---|---|---|
| Gene | Number of | Chromo- | acid | | | | 1000 | | Japanese |
| symbol | patients | some | mutation | Variation | Polyphen2 | SIFT | genomes | dbSNP | control |
| MME | 3 | chr3 | — | Splice | n.a | n.a | — | — | 0 |
| | 1 | chr3 | C621R | Missense | 1.0 (D) | 0.0 (D) | — | — | 0 |
| | 1 | chr3 | Q221X | Nonsense | 0.735 (P) | 0.0 (D) | — | — | 0 |
| FAT3 | 1 | chr11 | P2041H | Missense | 0.978 (D) | 0.89 (T) | — | — | 0 |
| | 1 | chr11 | C3776Y | Missense | 0.170 (B) | 0.82 (T) | — | — | 0 |
| SELRC1 | 1 | chr1 | R39W | Missense | 0.998 (D) | 0.01 (D) | — | — | 0 |
| | 1 | chr1 | D6G | Missense | 0.777 (P) | 0.04 (D) | — | — | 0 |
| CAD | 1 | chr2 | T166I | Missense | 0.006 (B) | 0.05 (T) | — | — | 0 |
| | 1 | chr2 | R168Q | Missense | 0.001 (B) | 0.41 (T) | — | — | 0 |
| | 1 | chr2 | R834H | Missense | 0.001 (B) | 0.09 (T) | — | — | 0 |
| | 1 | chr2 | L1653R | Missense | 0.467 (P) | 0.16 (T) | — | — | 0 |

SIFT score: Amino acid mutation with probabilities <0.05 are predicted to be deleterious.

A homozygous mutation in each of the three genes, i.e., MME gene, FAT3 gene, and SELRC1 gene, was shared by two or more families and there existed five cases with MME mutation, one case with homozygous nonsense mutation, one case with homozygous missense mutation, and three cases with homozygous splicing site mutation. There existed two cases with mutation in FAT3 gene and two cases with mutation in SELRC1 gene, and the mutations were all homozygous missense mutations.

Polyphen2 and SIFT in the table are each a prediction tool capable of determining whether a mutation is a pathogenic mutation, and Polyphen2 scores a mutation as 0.000 (maximum probability of benignity) to 0.999 (maximum probability of damaging). SIFT predicts a mutation with less than 0.05 to be deleterious.

### <Five Families Having MME Mutation>

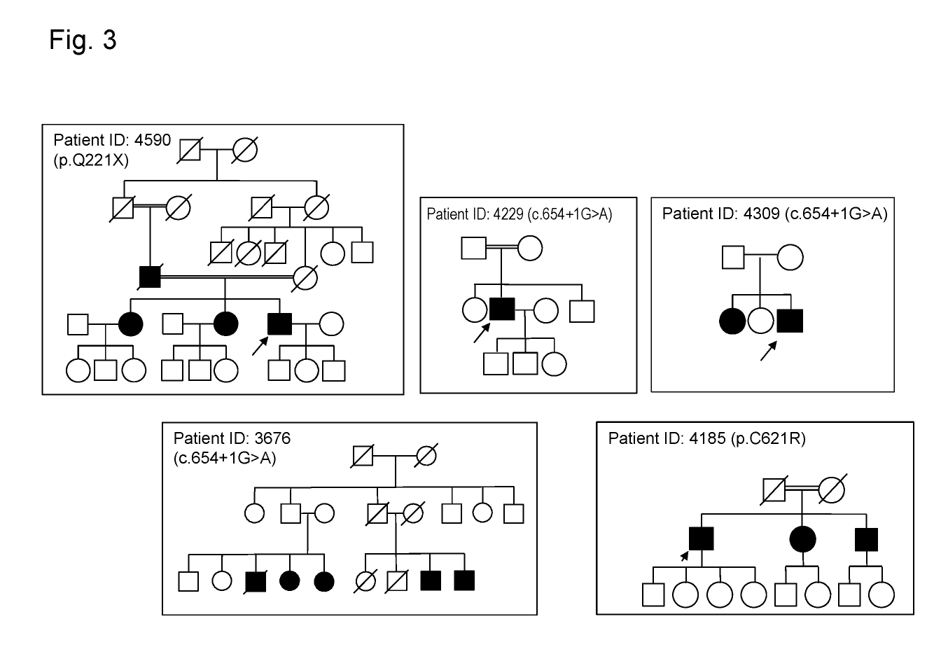

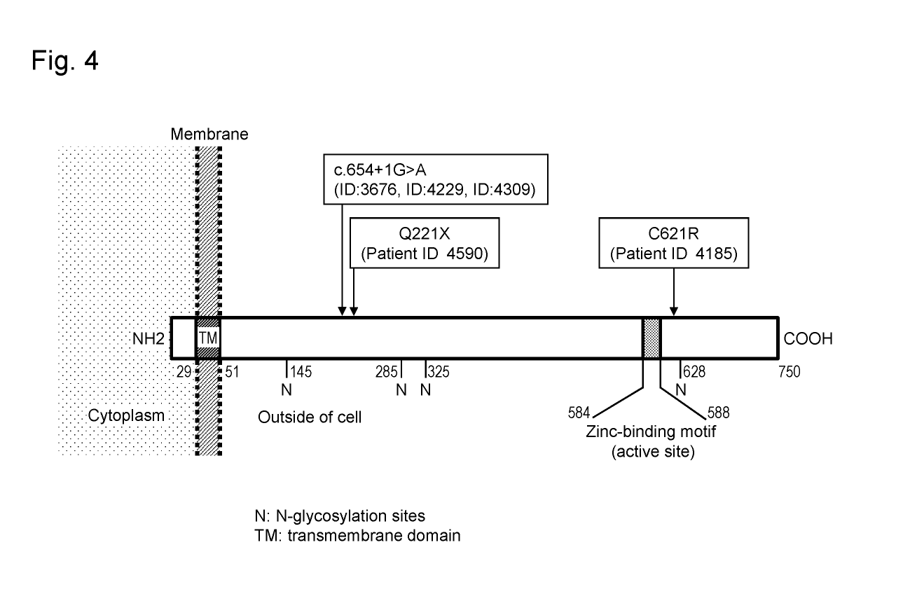

In the cases obtained here, the mutations in MME gene were a mutation on a splice donor site between exons 7 and 8 (c.654+1G>A) (a mutation of guanine to adenine at position 37341 of SEQ ID NO: 1) for three cases of patient ID: 4229, patient ID: 4309, and patient ID: 3676; a nonsense mutation on exon 8 (c.661C>T, p.Q221X) (a mutation of cytosine to thymine at position 39106 of SEQ ID NO: 1, a mutation of a glutamine residue to a termination codon at position 221 of SEQ ID NO: 2) for patient ID: 4590; and a missense mutation on exon 19 (c.1861T>C, p.C621R) (a mutation of thymine to cytosine at position 88926 of SEQ ID NO: 1, a mutation of a cysteine residue to an arginine residue at position 621 of SEQ ID NO: 2) for patient ID: 4185 (Table 11). The family histories of the five families having MME gene mutation are illustrated in FIG. 3, and a schematic of neprilysin encoded by MME gene and the mutation sites are illustrated in FIG. 4.

TABLE 11
Five families having MME gene mutation

| | | | | | | | Disease | | | |
|---|---|---|---|---|---|---|---|---|---|---|
| Gene | | Patient | Clinical | | | Onset | Duration | Family | Consan- | Initial |
| symbol | Mutation | ID | Diagnosis | Age | Sex | (years) | (years) | History | guinity | Symptom |
| MME | c.654 + 1G > A | 4229 | CMT2 | 49 | M | 36 | 13 | sporadic | + | run |
| | | | | | | | | | | disturbance |
| | c.654 + 1G > A | 4309 | AR-CMT2 | 56 | M | n.a | n.a | n.a | n.a | weakness |
| | c.654 + 1G > A | 3676 | CMT2 | 61 | M | 54 | 7 | AR. XD | − | gait |
| | | | CMTX | | | | | | | disturbance |
| | c.661C > T | 4590 | CMT1 | 58 | M | 48 | 10 | AR | + | gait |
| | p.Q221X | | | | | | | | | disturbance |
| | c.1861T > C | 4185 | AR-CMT2 | 63 | M | 50 | 13 | AR | + | gait |
| | p.C621R | | | | | | | | | disturbance |

| | | | | | | Nerve conduction | | | |
|---|---|---|---|---|---|---|---|---|---|
| | | | | | | study | | | |
| | | | | | | Median nerve | | | |
| Gene | | MMT | Sensory | CNS | | DL | dCMAP | MCV | Nerve |
| symbol | Mutation | (TA) | Disturbance | involvement | Type | (ms) | (mV) | (m/s) | biopsy |
| MME | c.654 + 1G > A | 2 | + | — | axonal | 4.3 | 4.5 | 42.8 | n.a |
| | c.654 + 1G > A | 1 | + | — | axonal | 3.6 | 10.0 | 53 | n.a |
| | c.654 + 1G > A | 4 | + | — | axonal | n.a | n.a | n.A | n.a |
| | c.661C > T | 3 | + | — | axonal | 4.3 | 4.5 | 37.4 | n.a |
| | p.Q221X | | | | | | | | |
| | c.1861T > C | 4 | + | — | axonal | 6.3 | 3.2 | 45.5 | intermediate |
| | p.C621R | | | | | | | | deficit of |
| | | | | | | | | | myelinated |
| | | | | | | | | | fibers, |
| | | | | | | | | | Onion bulb |
| | | | | | | | | | (—) |

In segregation analysis for patient ID: 3676 (IV-3) using Sanger method (detection of a mutation in the sequence GTAATTCATgtaagtt (SEQ ID NO: 17, the amino acid sequence contains VIH)), as an example, a homozygous mutation of c.654+1G>A was found in MME of his female cousin (IV-9), who was an affected individual, and a heterozygous mutation of c.654+1G>A (indication of being a carrier) was found in MME of his sons (V-1) and (V-5), who were healthy individuals, and thus cosegregation in the family was confirmed (FIG. 5).

According to clinical information on five cases with MME mutation, the age of onset was 35 years old or older in all of the five cases and parents had consanguineous marriage in three cases. In three cases of four cases in which a nerve conduction test was conducted, the motor nerve conduction velocity in the median nerve was 38 m/s or higher, and the cases were determined to be an axonal type. In another case (patient ID: 4590), the motor nerve conduction velocity in the median nerve was 37.4 m/s, which indicated an intermediate type, and both of the tibial nerve CMAP (compound muscle action potential) and the sural nerve SNAP (sensory nerve action potential) in the lower limb could not be elicited, and the patient was clinically diagnosed as axonal motor sensory neuropathy. Neurological symptoms other than neuropathy were not found for all four cases.

### <Two Families Having FAT3 Mutation>

In the cases obtained here, the mutations in FAT3 gene found were a missense mutation on exon 9 (c.6122C>A, p.P2041H) (a mutation of cytosine to adenine at position 484856 of SEQ ID NO: 3, a mutation of a proline residue to a histidine residue at position 2041 of SEQ ID NO: 4) for patient ID: 3743; and a missense mutation on exon 18 (c.11327G>A, p.C3776Y) (a mutation of guanine to adenine at position 530415 of SEQ ID NO: 3, a mutation of a cysteine residue to a tyrosine residue at position 3776 of SEQ ID NO: 4) for patient ID: 3887 (Table 12).

TABLE 12
Two families having FAT3 gene mutation

| | | | | | | | Disease | | | |
|---|---|---|---|---|---|---|---|---|---|---|
| Gene | | Patient | Clinical | | | Onset | Duration | Family | Consan- | Initial |
| symbol | Mutation | ID | Diagnosis | Age | Sex | (years) | (years) | History | guinity | Symptom |
| FAT3 | c.6122C > A | 3743 | CMT2 | 25 | F | 2 | 23 | AR | +second | gait |
| | p.P2041H | | DSS | | | | | | cousin | disturbance |
| | c.11327G > A | 3887 | CMT2 | 62 | M | 35 | 30 | AR | + | muscle |
| | p.C3776Y | | | | | | | | | cramp |

| | | | | | | Nerve conduction | | | |
|---|---|---|---|---|---|---|---|---|---|
| | | | | | | study | | | |
| | | | | | | Median nerve | | | |
| Gene | | MMT | Sensory | CNS | | DL | dCMAP | MCV | Nerve |
| symbol | Mutation | (TA) | Disturbance | involvement | Type | (ms) | (mV) | (m/s) | biopsy |
| FAT3 | c.6122C > A | n.a | + | +swallowing | unknown | ND | ND | ND | n.a |
| | p.P2041H | (severe) | | disturbance, | | | | | |
| | | | | tongue atrophy | | | | | |
| | c.11327G > A | 0 | + | +swallowing | unknown | n.a | n.a | n.a | high reduction |
| | p.C3776Y | | | disturbance, | | | | | of |
| | | | | tongue atrophy, | | | | | myelinated and |
| | | | | hoarseness | | | | | unmyelinated |
| | | | | | | | | | fibers |

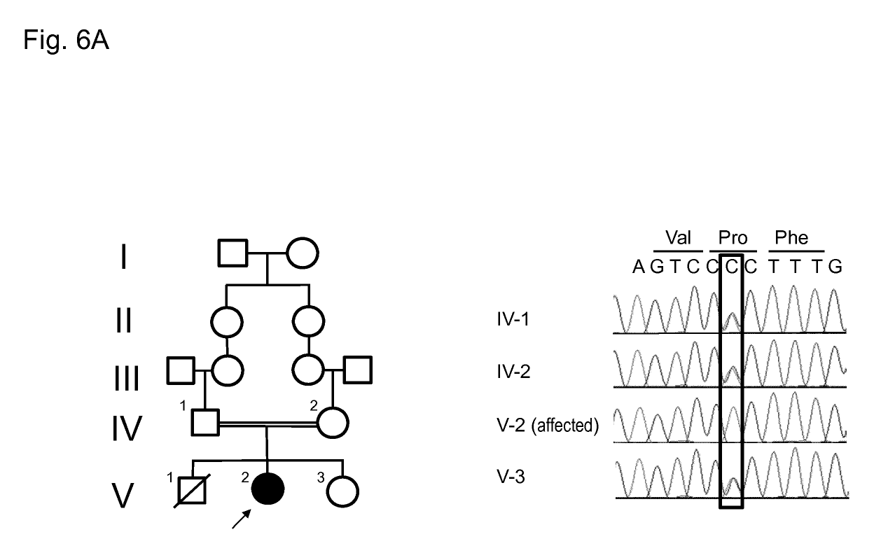

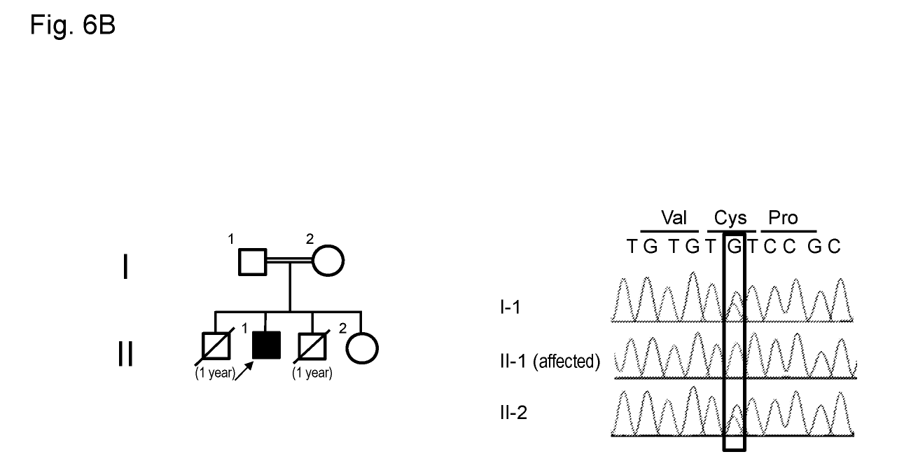

In both cases, parents had consanguineous marriage and the muscle strength of the distal part of the lower limb was highly weakened, and sensory disturbance was found. In a nerve conduction study for patient ID: 3743, the motor and sensory nerve in the limbs was not elicited at all. On sural nerve biopsy of patient ID: 3887, a remarkable decrease in the density of myelinated and unmyelinated fibers and chronic axonal degeneration with poor reproduction were observed. In both cases, surprisingly, the patient was presenting with the similar cranial nerve symptoms such as dysphagia and tongue atrophy. In segregation analysis for patient ID: 3743 using Sanger method (detection of a mutation in the sequence AGTCCCCTTTG (SEQ ID NO: 18, the amino acid sequence contains VPF), a heterozygous mutation of c.6122C>A, p.P2041H (indication of being a carrier) was found in FAT3 of each of his father (IV-1), his mother (IV-2), and his younger sister (V-3), who were each a healthy individual, and thus cosegregation in the family was confirmed (FIG. 6A). In segregation analysis for patient ID: 3887 using Sanger method (detection of a mutation in the sequence TGTGTGTCCGC (SEQ ID NO: 19, the amino acid sequence contains VCP), a heterozygous mutation of c.11327G>A, p.C3776Y (indication of being a carrier) was found in FAT3 of his father (I-1) and his younger sister (II-2), who were each a healthy individual, and thus cosegregation in the family was confirmed (FIG. 6B).

### <Two Cases Having SELRC1 Mutation>

In the cases obtained here, the mutations in SELRC1 gene found were a missense mutation on exon 2 (c.115C>T, p.R39W) (a mutation of cytosine to thymine at position 5508 of SEQ ID NO: 5, a mutation of an arginine residue to a tryptophan residue at position 39 of SEQ ID NO: 6) for patient ID: 4348; and a missense mutation on exon 1 (c.17A>G, p.D6G) (a mutation of adenine to guanine at position 57 of SEQ ID NO: 5, a mutation of an aspartic acid residue to a glycine residue at position 6 of SEQ ID NO: 6) for patient ID: 4040 (Table 13).

TABLE 13
Two families having SELRC1 gene mutation

| | | | | | | | Disease | | | |
|---|---|---|---|---|---|---|---|---|---|---|
| Gene | | Patient | Clinical | | | Onset | Duration | Family | Consan- | Initial |
| symbol | Mutation | ID | Diagnosis | Age | Sex | (years) | (years) | History | guinity | Symptom |
| SELRC1 | c.115C > T | 4348 | CMT2 | 16 | F | 4 | 12 | sporadic | − | gait |
| | p.R39W | | | | | | | | | disturbance |
| | c.17A > G | 4040 | CMT2 | 57 | M | <10 | 50 | AR | + | foot |
| | p.D6G | | | | | | | | | deformity |

| | | | | | | Nerve conduction | | | |
|---|---|---|---|---|---|---|---|---|---|
| | | | | | | study | | | |
| | | | | | | Median nerve | | | |
| Gene | | MMT | Sensory | CNS | | DL | dCMAP | MCV | Nerve |
| symbol | Mutation | (TA) | disturbance | involvement | Type | (ms) | (mV) | (m/s) | biopsy |
| SELRC1 | c.115C > T | n.a | + | cerebellar | axonal | n.a | n.a | 55 | significant reduction of |
| | p.R39W | | | ataxia, | | | | | myelinated fibers, onion |
| | | | | intellectual | | | | | bulb (+), axonal |
| | | | | disability | | | | | degeneration (+) |
| | c.17A > G | 0 | + | cerebellar | axonal | 3.75 | 8.2 | 52.1 | significant reduction of |
| | p.D6G | | | ataxia | | | | | large myelinated fibers, |
| | | | | | | | | | mild demyelination |

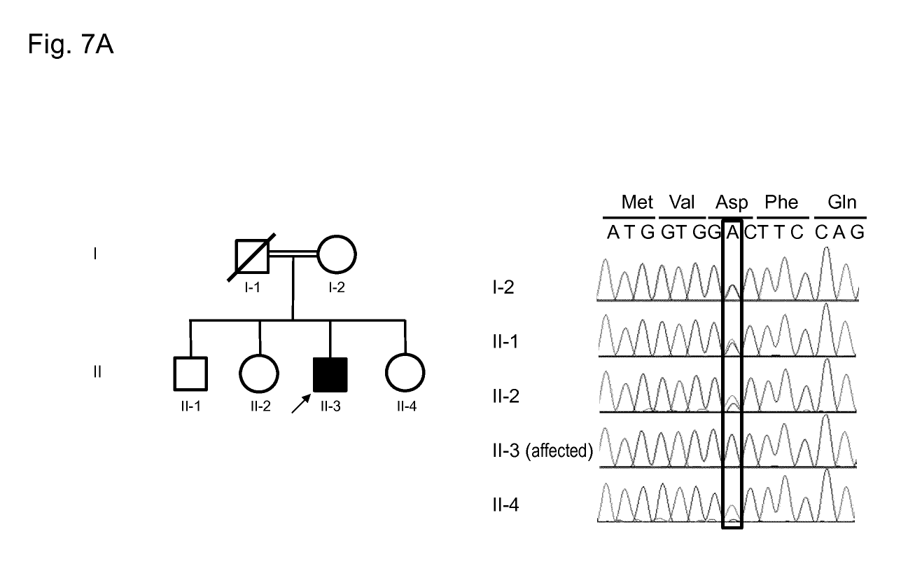

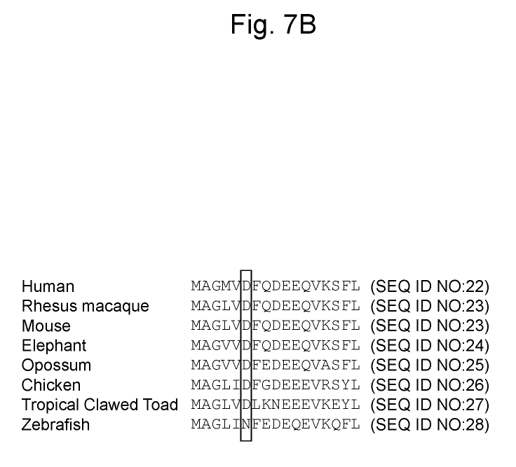

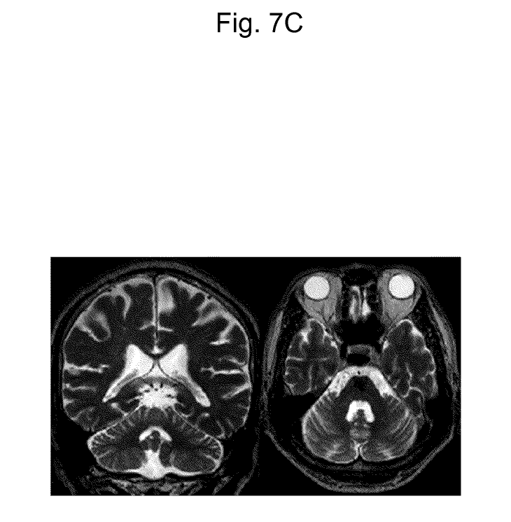

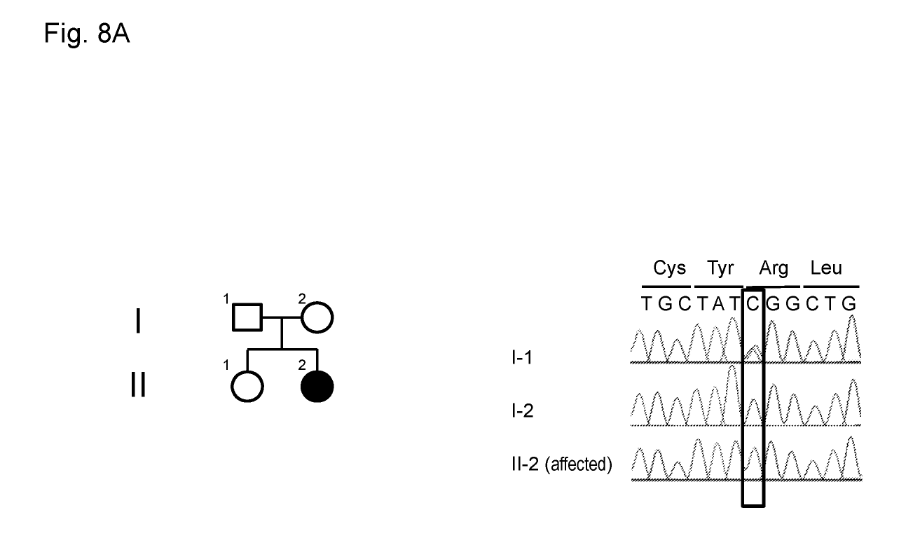

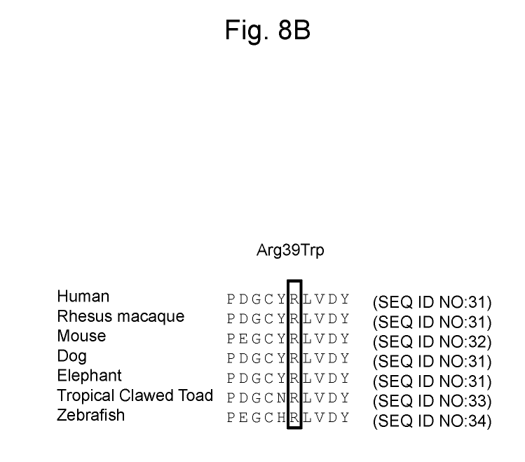

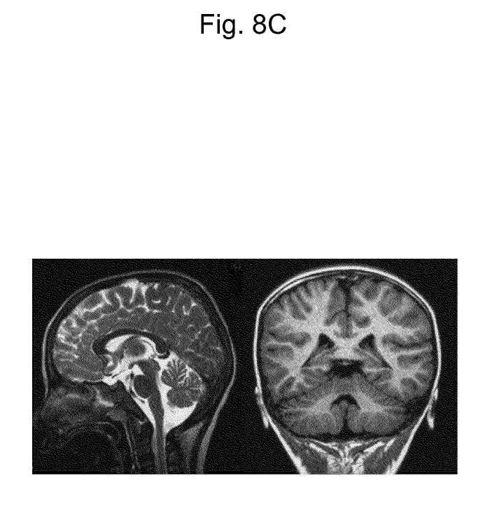

The parents of patient ID: 4040 had consanguineous marriage. In both of the two cases, the patients were clinically diagnosed as axonal CMT2 before the genetic test, and the onset had occurred in the infant stage in both cases. In a nerve conduction study, the motor nerve conduction velocity in the median nerve was over 50 m/s for both cases, and they were classified as an axonal type. On sural nerve biopsy, a significant reduction of large myelinated fibers was observed for both cases. The onion bulb formation and axonal degeneration were found for patient ID: 4348. The number of large myelinated fibers was significantly reduced to 0 to 2 fibers per nerve bundle for patient ID: 4040. Surprisingly, both patients had similar phenotypes characterized by mild cerebellar ataxia and cerebellar atrophy on MRI (FIGS. 7C and 8C). On single photon emission computed tomography for patient ID: 4040, mild decreased cerebellum blood flow was observed.

In segregation analysis for patient ID: 4040 using Sanger method (detection of a mutation in the nucleotide sequence ATGGTGGACTTCCAG (SEQ ID NO: 20), the amino acid sequence MVDFQ (SEQ ID NO: 21)), a heterozygous mutation of c.17A>G, p.D6G was found in SELRC1 of each of his mother (I-2), his older brother (II-1), and his older sister (II-2), each an healthy individual, and the mutation was not found for his younger brother (II-4), and thus cosegregation in the family was confirmed (FIG. 7A).

FIG. 7B shows interspecies comparison of a partial sequence containing Asp6 in the amino acid sequence encoded by SELRC1 gene (SEQ ID NO: 6). The sequences before and after Asp, the sixth amino acid of a protein encoded by SELRC1 gene, are relatively conserved among species, which suggests that the conservation of the sequence is likely to be important for the function of the protein.

In segregation analysis for patient ID: 4348 using Sanger method (detection of a mutation in the sequence TGCTATCGGCTG (SEQ ID NO: 29), the amino acid sequence CYRL (SEQ ID NO: 30)), a heterozygous mutation of c.115C>T, p.R39W (indication of being a carrier) was found in SELRC1 of his farther (I-1), a healthy individual, and the mutation was not found for his mother (I-2), and thus cosegregation in the family was confirmed (FIG. 8A).

FIG. 8B shows interspecies comparison of a partial sequence containing Arg39 in the amino acid sequence encoded by SELRC1 gene (SEQ ID NO: 6). The sequences before and after Arg, the 39th amino acid of a protein encoded by SELRC1 gene, are relatively conserved among species, which suggests that the conservation of the sequence is likely to be important for the function of the protein.

### <Two Cases Having CAD Mutation>

In each of two cases of patient ID: 4539 and patient ID: 3353 obtained here, the translated region of CAD (carbamoyl-phosphate synthetase 2, aspartate transcarbamylase, and dihydroorotase) gene had two heterozygous missense mutations, and the mutations were suspected to be compound heterozygous mutation (Table 14).

TABLE 14
Two families CAD gene mutation

| | | | | | | | | Disease | | | |
|---|---|---|---|---|---|---|---|---|---|---|---|
| Gene | | | Patient | Clinical | | | Onset | Duration | Family | Consan- | Initial |
| symbol | Mutation | Genotype | ID | Diagnosis | Age | Sex | (years) | (years) | History | guinity | Symptom |
| CAD | T166I | CH | 4539 | AR-CMT2 | 60 | F | 39 | 21 | AR | − | gait |
| | R168Q | | | | | | | | | | disturbance |
| | R834H | CH | 3353 | AR-CMT2 | 59 | M | 56 | 3 | AR | + | gait |
| | L1653R | | | | | | | | | | disturbance |

| | | | | | | Nerve conduction | | | |
|---|---|---|---|---|---|---|---|---|---|
| | | | | | | study | | | |
| | | | | | | Median nerve | | | |
| Gene | | MMT | Sensory | CNS | | DL | dCMAP | MCV | Sural nerve |
| symbol | Mutation | (TA) | disturbance | involvement | Type | (ms) | (mV) | (m/s) | pathology |
| CAD | T166I | 4 | + | facial | axonal | 3.4 | 12.8 | 58.7 | n.a |
| | R168Q | | | weakness | | | | | |
| | R834H | 2 | + | — | axonal | 5.1 | 8.3 | 46.9 | n.a |
| | L1653R | | | | | | | | |

Patient ID: 4539 had heterozygous mutations of c.497C>T, p.T166I (a mutation of cytosine to thymine at position 5131 of SEQ ID NO: 15, a mutation of a threonine residue to an isoleucine residue at position 166 of SEQ ID NO: 16) and c.503G>A, p.R168Q (a mutation of guanine to adenine at position 5137 of SEQ ID NO: 15, a mutation of an arginine residue to a glutamine residue at position 168 of SEQ ID NO: 16) in CAD gene. Patient ID: 3353 had heterozygous mutations of c.2501G>A, p.R834H (a mutation of guanine to adenine at position 14691 of SEQ ID NO: 15, a mutation of an arginine residue to a histidine residue at position 834 of SEQ ID NO: 16) and c.4958T>G, p.L1653R (a mutation of thymine to guanine at position 21139 of SEQ ID NO: 15, a mutation of a leucine residue to an arginine residue at position 1653 of SEQ ID NO: 16).

In the family history of patient ID: 4539, the parents were not affected and his younger brother was diagnosed as CMT, which suggested autosomal recessive inheritance. In the family history of patient ID: 3353, the parents had consanguineous marriage, which suggested autosomal recessive inheritance. In both of the two cases, the onset had occurred at or after the adult stage, and the patients were clinically diagnosed as axonal motor sensory neuropathy on the basis of findings in a nerve conduction study. Segregation analysis was not performed for the two cases. A protein encoded by CAD is a multienzyme complex having the first three enzymatic functions in the de novo biosynthesis pathway of pyrimidine nucleotide (C: carbamoyl-phosphate synthetase (CPS II), A: aspartate carbamoyltransferase, D: dihydroorotase). Although the function of CAD in peripheral nervous system is unclear, CAD is known to be present in the cytoplasm and closely associated with cellular proliferative potential, and to have a high activity in normal cells with active division and proliferation in thymus, testis, spleen, etc., and in various tumor cells.

### <Four Genes Having Homozygous Nonsense Mutation>

The present inventors further found four genes as candidate genes for AR-CMT having a homozygous nonsense mutation: ABCC3 (ATP-binding cassette, subfamily C (CFTR/MRP), member 3), ANKRD7 (ankyrin repeat domain 7), CNGA4 (cyclic nucleotide-gated channel α4), and COL6A6 (collagen, type VI, α6) (Table 15).

TABLE 15

| | | | | | | | | | | Allele Frequency in | | | | |
|---|---|---|---|---|---|---|---|---|---|---|---|---|---|---|
| | | | | | | | | | | Japanese control | | | | |
| | | | | | | | | | | Frequency | | | | Number |
| | Number | | | Nucle- | Amino | | | 1000 | | of alter- | Genotype count | | | of |
| Gene | of | Chromo- | | otide | acid | Geno- | Varia- | ge- | | native | Ref/ | Ref/ | Alt/ | Samples |
| symbol | patients | some | Position | change | change | type | tion | nomes | dbSNP | allele | Ref | Alt | Alt | Covered |
| ABCC3 | 1 | chr17 | 48764928 | C > T | R1438X | Homo | Nonsense | — | — | 0 | 0 | 0 | 0 | |
| ANKRD7 | 1 | chr7 | 117874773 | G > T | E105X | Homo | Nonsense | — | — | 0.004 | 761 | 6 | 0 | 767 |
| CNGA4 | 1 | chr11 | 6261928 | C > T | R302X | Homo | Nonsense | — | — | 0.012* | 391 | 1 | 0 | 392 |
| COL6A6 | 1 | chr3 | 130282181 | C > T | Q112X | Homo | Nonsense | — | — | 0.02 | 299 | 1 | 0 | 300 |

*The allele frequency of CNGA4 includes in-house disease control database (N = 388).

In the cases obtained, the mutation found in ABCC3 gene was a homozygous nonsense mutation on chromosome 17 (48764928 C>T R1438X) (a mutation of cytosine to thymine at position 52711 of SEQ ID NO: 7, a mutation of an arginine residue to a termination codon at position 1438 of SEQ ID NO: 8).

In the cases obtained, the mutation found in ANKRD7 gene was a homozygous nonsense mutation on chromosome 7 (117874773 G>T E105X) (a mutation of guanine to thymine at position 10281 of SEQ ID NO: 9, a mutation of a glutamic acid residue to a termination codon at position 105 of SEQ ID NO: 10).

In the cases obtained, the mutation found in CNGA4 gene was a homozygous nonsense mutation on chromosome 11 (6261928 C>T R302X) (a mutation of cytosine to thymine at position 5205 of SEQ ID NO: 11, a mutation of an arginine residue to a termination codon at position 302 of SEQ ID NO: 12).

In the cases obtained, the mutation found in COL6A6 gene was a homozygous nonsense mutation on chromosome 3 (130282181 C>T Q112X) (a mutation of cytosine to thymine at position 3004 of SEQ ID NO: 13, a mutation of a glutamine residue to a termination codon at position 112 of SEQ ID NO: 14).

All of these mutations had not been registered in known databases, and relation to the onset of CMT was suggested.

All publications, patents, and patent applications referred to herein are directly incorporated herein as reference.

1. A method for acquiring data for diagnosis of autosomal recessive inherited Charcot-Marie-Tooth disease (CMT), wherein a mutation(s) in MME (membrane metallo-endopeptidase) gene, FAT3 (FAT tumor suppressor homolog 3) gene, and/or SELRC1 (Sell repeat containing 1) gene in a biological sample are/is detected.

2. A method for acquiring data for diagnosis of Charcot-Marie-Tooth disease, wherein a mutation in a DNA in a biological sample is detected, and the mutation is one or more mutations in the nucleotide sequence represented by SEQ ID NO: 1, 3, or 5.

3. The method according to claim 1, wherein the mutation is any non-synonymous mutation of missense mutation, nonsense mutation, and frameshift mutation.

4. A method for acquiring data for diagnosis of Charcot-Marie-Tooth disease comprising:
determining a nucleotide sequence of MME gene in a sample; and
comparing the sequence with a nucleotide sequence represented by SEQ ID NO: 1 to determine the presence or absence of a mutation.

5. The method according to claim 4, wherein the mutation is one or more of a mutation on a splice donor site between exons 7 and 8 in MME gene (c.654+1G>A) (a mutation of guanine to adenine at position 37341 of SEQ ID NO: 1), a nonsense mutation on exon 8 in MME gene (c.661C>T, p.Q221X) (a mutation of cytosine to thymine at position 39106 of SEQ ID NO: 1, a mutation of a glutamine residue to a termination codon at position 221 of SEQ ID NO: 2), and a missense mutation on exon 19 in MME gene (c.1861T>C, p.C621R) (a mutation of thymine to cytosine at position 88926 of SEQ ID NO: 1, a mutation of a cysteine residue to an arginine residue at position 621 of SEQ ID NO: 2).

6. A method for acquiring data for diagnosis of Charcot-Marie-Tooth disease comprising:
determining a nucleotide sequence of FAT3 gene in a sample; and
comparing the sequence with a nucleotide sequence represented by SEQ ID NO: 3 to determine the presence or absence of a mutation.

7. The method according to claim 6, wherein the mutation is one or more of a missense mutation on exon 9 in FAT3 gene (c.6122C>A, p.P2041H) (a mutation of cytosine to adenine at position 484856 of SEQ ID NO: 3, a mutation of a proline residue to a histidine residue at position 2041 of SEQ ID NO: 4) and a missense mutation on exon 18 in FAT3 gene (c.11327G>A, p.C3776Y) (a mutation of guanine to adenine at position 530415 of SEQ ID NO: 3, a mutation of a cysteine residue to a tyrosine residue at position 3776 of SEQ ID NO: 4).

8. A method for acquiring data for diagnosis of Charcot-Marie-Tooth disease comprising:
determining a nucleotide sequence of SELRC1 gene in a sample; and
comparing the sequence with a nucleotide sequence represented by SEQ ID NO: 5 to determine the presence or absence of a mutation.

9. The method according to claim 8, wherein the mutation is one or more of a missense mutation on exon 2 in SELRC1 gene (c.115C>T, p.R39W) (a mutation of cytosine to thymine at position 5508 of SEQ ID NO: 5, a mutation of an arginine residue to a tryptophan residue at position 39 of SEQ ID NO: 6) and a missense mutation on exon 1 in SELRC1 gene (c.17A>G, p.D6G) (a mutation of adenine to guanine at position 57 of SEQ ID NO: 5, a mutation of an aspartic acid residue to a glycine residue at position 6 of SEQ ID NO: 6).

10. A primer or probe for detection of autosomal recessive inherited CMT, being a nucleic acid consisting of a nucleotide sequence represented by any of SEQ ID NOs: 1, 3 and 5 or a partial nucleic acid thereof.

11. A DNA chip for detection of autosomal recessive inherited CMT, comprising the probe according to claim 10.

12. A kit for detection of a mutation in MME gene, FAT3 gene, and/or SELRC1 gene in a biological sample to be used in the method according to claim 1.

13. The method according to claim 2, wherein the mutation is any non-synonymous mutation of missense mutation, nonsense mutation, and frameshift mutation.
